SEQUENCE LISTING (2 sequences)

SEQ ID NO: 1 - DNA, 22 bp, Artificial Sequence
  primer
cgggaattctgcaggctcagag

SEQ ID NO: 2 - DNA, 30 bp, Artificial Sequence
  primer
gggaattcgagctccgcggcagcctgacca

(19) United States
(12) Patent Application Publication
(10) Pub. No.: US 2005/0262578 A1
(43) Pub. Date: Nov. 24, 2005

(54) Non-human animal models of atherosclerosis and methods of use thereof

(76) Inventors: Robert E. Pitas, Albany, CA (US); Matthias Schneider, San Francisco, CA (US); Thomas L. Innerarity, Lafayette, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/956,790

(22) Filed: Oct. 1, 2004

Related U.S. Application Data
(60) Provisional application No. 60/600,133, filed on Aug. 9, 2004.
(60) Provisional application No. 60/508,400, filed on Oct. 3, 2003.

(51) Int. Cl. A01K 67/027

(52) U.S. Cl. 800/9; 800/14

(57) ABSTRACT

The present invention provides transgenic, non-human animals comprising a transgene that encodes apolipoprotein(a), which animal exhibits an atherosclerotic phenotype. The present invention further provides transgenic, non-human animals comprising a transgene that encodes apolipoprotein(a) and a transgene that encodes apolipoprotein B-100, which animal exhibits an atherosclerotic phenotype. The present invention further provides methods of identifying agents that reduce atherosclerosis, as well as agents identified by the methods.

### CROSS-REFERENCE

This application claims the benefit of U.S. Provisional Patent Application No. 60/508,400, filed Oct. 3, 2003, and U.S. Provisional Patent Application No. 60/600,133, filed Aug. 9, 2004, which applications are incorporated herein by reference in its entirety.

### STATEMENT REGARDING FEDERALLY SPONSORED RESEARCH

The U.S. government may have certain rights in this invention, pursuant to grant no. HL-41633 awarded by the National Institutes of Health.

### FIELD OF THE INVENTION

The present invention is in the field of non-human transgenic animal models of atherosclerosis.

### BACKGROUND OF THE INVENTION

Lipoprotein(a) (Lp(a)) is composed of apolipoprotein(a) (apo(a)), a plasminogen-like glycoprotein, covalently linked to apoB-100 of low density lipoproteins (LDL). Apo(a) contains a variable number of copies of a kringle-like domain that has approximately 75% identity with kringle IV of plasminogen, a single kringle V-like domain, and a catalytically inactive protease-like domain similar to plasminogen. Epidemiological studies have shown that Lp(a) mass plasma concentrations above 30 mg/dl are associated with increased risk of atherosclerosis, myocardial infarction, and cerebral stroke.

Although several pathogenic roles for Lp(a) have been proposed, its physiological and pathological functions remain elusive, in part because of the lack of animal models expressing high levels of Lp(a). Lawn et al. ((1992) Nature 360:670-672) found small fatty-streak lesions in the proximal ascending aorta of transgenic mice expressing low levels of apo(a). However, in that model, the apo(a) does not covalently associate with mouse apoB and hence the plasma does not contain true Lp(a). Transgenic mice coexpressing human apo(a) and apoB-100 formed bonafide Lp(a) (Sanan et al. (1998) Proc. Natl. Acad. Sci. USA 95:4544-4549) but had no increase in atherosclerosis compared to transgenic mice expressing apoB-100 alone. However, the mean plasma level of Lp(a) was only 27 mg/dl, and the mice had high levels of LDL, which might have resulted in a high background level of atherosclerosis (Sanan et al. (1998) supra). In addition, the variable genetic background of the different mice might have confounded interpretation of the data. Others (Sun et al. (2002) J. Biol. Chem. 277:47486-47492) expressed apo(a) in wild-type and Watanabe heritable hyperlipidemic transgenic rabbits and fed them a hypercholesterolemic or chow diet, respectively. The Watanabe rabbits expressing Lp(a) had significantly more atherosclerosis than nontransgenic littermates, and the advanced lesions had calcification. However, the mean plasma level of Lp(a)-like particles in the rabbits was only 15 mg/dl, and most of the apo(a) in rabbits is not covalently linked to apoB-100 of LDL. A limitation of all of the animal models designed to examine the metabolism of Lp(a) and the pathological role of Lp(a) has been the expression of apo(a) at levels that have few pathological consequences in humans or lack of formation of Lp(a) in which the apo(a) is covalently bound to apoB-100.

There is a need in the art for animal models of atherosclerosis that are suitable to serve as models for atherosclerosis in humans. The present invention addresses this need.

Literature

Schneider et al. (Nov. 5, 2002) Circulation Scientific Sessions Abstracts Suppl. Vol. 106, #19; Lawn et al. (1992) Nature 360:670-672; Sanan et al. (1998) Proc. Natl. Acad. Sci. USA 95:4544-4549; Sun et al. (2002) J. Biol. Chem. 277:47486-47492; U.S. Pat. No. 6,512,161; U.S. patent Publication No. 2004/0138164; U.S. patent Publication No. 2003/0119766.

### SUMMARY OF THE INVENTION

The present invention provides transgenic, non-human animals comprising a transgene that encodes apolipoprotein(a), which animal exhibits an atherosclerotic phenotype. The present invention farther provides transgenic, non-human animals comprising a transgene that encodes apolipoprotein(a) and a transgene that encodes apolipoprotein B-100, which animal exhibits an atherosclerotic phenotype. The present invention further provides methods of identifying agents that reduce atherosclerosis, as well as agents identified by the methods.

### BRIEF DESCRIPTION OF THE DRAWINGS

FIG. 1 is a schematic representation of a construct for liver-specific expression of apo(a).

FIG. 2 depicts analysis of genomic DNA from apo(a) transgenic mice.

FIG. 3 depicts Northern blot analysis of expression of apo(a) in various tissues of an apo(a) transgenic mouse.

FIG. 4 depicts an analysis of plasma levels of apo(a) in apo(a) transgenic mice.

FIG. 5 depicts lipoprotein profiles of non-transgenic and apo(a) transgenic mice.

FIG. 6 depicts apo(a)-dependent formation of Lp(a) in vitro.

FIG. 7 depicts formation of Lp(a) in vivo in mice expressing high or low levels of apo(a) together with human apoB-100.

FIGS. 8A and 8B depict lipoprotein profiles of mice expressing human apoB-100 or high levels of Lp(a).

FIG. 9 depicts distribution of apo(a) and human apoB-100 plasma of transgenic mice expressing either or both proteins.

FIGS. 10A and 10B depict an analysis of oxidized phospholipids on captured human apoB-100-containing lipoproteins in transgenic mouse plasma.

FIGS. 11A-D depict an analysis of oxidized phospholipids and apo(a) in Lp(a) that contains human or mouse apoB-100.

FIG. 12 depicts the mean lesion area in mice after 39 weeks on a high fat, high cholesterol diet.

FIG. 13 depicts aortic lesions in mice after 39 weeks on a high fat, high cholesterol diet.

### DEFINITIONS

The term “transgene” is used herein to describe genetic material which has been or is about to be artificially inserted into the genome of a non-human animal, and particularly into a cell of a living non-human mammal.

The term “transformation” refers to a permanent or transient genetic change induced in a cell following the incorporation of new DNA (i.e. DNA exogenous to the cell). Where the cell is a mammalian cell, a permanent genetic change is generally achieved by introduction of the DNA into the genome of the cell, such that the cell is said to be “genetically modified.”

The term “ES cell,” or “embryonic stem cell,” as used herein, refers to pluripotent embryonic stem cells and to such pluripotent cells in the very early stages of embryonic development, including but not limited to cells in the blastocyst stage of development.

The term “construct” refers to a recombinant nucleic acid, generally recombinant DNA, that has been generated for the purpose of the expression of a specific nucleotide sequence(s), or is to be used in the construction of other recombinant nucleotide sequences.

The term “operably linked” refers to a functional connection between a DNA sequence and a regulatory or a control sequence(s), e.g., a DNA sequence and a regulatory sequence(s) are connected in such a way as to permit gene expression when the appropriate factor (e.g., transcriptional activator proteins) are bound to the regulatory sequence(s).

The term “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein.

The term “genomic sequence” refers to a sequence having non-contiguous open reading frames, where introns interrupt the protein coding regions. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a transgenic non-human animal” includes a plurality of such animals and reference to “the transgene” includes reference to one or more transgenes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides transgenic, non-human animals comprising a transgene that encodes apolipoprotein(a), which animal may exhibit an atherosclerotic phenotype. The present invention further provides transgenic, non-human animals comprising a transgene that encodes apolipoprotein(a) and a transgene that encodes apolipoprotein B-100, which animal exhibits an atherosclerotic phenotype. The present invention further provides methods of identifying agents that reduce atherosclerosis, as well as agents identified by the methods.

Transgenic Non-Human Animals

The present invention provides transgenic non-human animals that comprise a transgene encoding apolipoprotein(a) (apo(a)). The present invention further provides transgenic non-human animals that comprise a transgene encoding apo(a) and a transgene encoding apolipoprotein B-100 (apoB-100). A subject transgenic animal exhibits an atherosclerotic phenotype, and is useful as a model of atherosclerosis. A subject transgenic animal is useful in research applications, for studying, e.g., the effects of diet and other factors on atherosclerosis. A subject transgenic animal is also useful for drug screening, e.g., to identify agents that treat atherosclerosis, that reduce the risk that an individual will develop atherosclerosis, etc.

In some embodiments, a subject transgenic non-human animal comprises a transgene that encodes apo(a). Such animals are useful for crossing with apoB-100 transgenic animals, to generate double transgenic animals The apo(a) transgenic animals are useful in research applications and for generating apo(a)/apoB-100 double transgenic animals. In other embodiments, a subject transgenic non-human animal comprises both a transgene that encodes apo(a) and a transgene that encodes apoB-100. The apo(a)/apoB-100 double transgenic animals are models of atherosclerosis, and thus are useful in research applications and for identifying agents that treat atherosclerosis. In a subject transgenic non-human animal that comprises an apo(a) transgene, the apo(a) transgene is expressed, and the apo(a) protein encoded by the transgene is synthesized in the animal. In a subject transgenic non-human animal that comprises an apo(a) transgene and an apoB-100 transgene, the apo(a) and apoB-100 transgenes are expressed, and the apo(a) and apoB-100 proteins encoded by the transgenes are synthesized in the animal.

In some embodiments, a subject transgenic animal has a plasma lipoprotein(a) (Lp(a)) level that is in excess of 30 mg/dL, e.g., a subject transgenic animal has a plasma Lp(a) level of from about 30 mg/dL to about 1500 mg/dL, e.g., a subject transgenic animal has a plasma Lp(a) level of from about 30 mg/dL to about 40 mg/dL, from about 40 mg/dL to about 50 mg/dL, from about 50 mg/dL to about 100 mg/dL, from about 100 mg/dL to about 150 mg/dL, from about 150 mg/dL to about 200 mg/dL, from about 200 mg/dL to about 250 mg/dL, from about 250 mg/dL to about 300 mg/dL, from about 300 mg/dL to about 350 mg/dL, from about 350 mg/dL to about 400 mg/dL, from about 400 mg/dL to about 450 mg/dL, from about 450 mg/dL to about 500 mg/dL, from about 500 mg/dL to about 550 mg/dL, from about 550 mg/dL to about 600 mg/dL, from about 600 mg/dL to about 650 mg/dL, from about 650 mg/dL to about 700 mg/dL, from about 700 mg/dL to about 750 mg/dL, from about 750 mg/dL to about 800 mg/dL, from about 800 mg/dL to about 900 mg/dL, from about 900 mg/dL to about 1000 mg/dL, from about 1000 mg/dL to about 1100 mg/dL, from about 1100 mg/dL to about 1200 mg/dL, from about 1200 mg/dL to about 1300 mg/dL, from about 1300 mg/dL to about 1400 mg/dL, or from about 1400 mg/dL to about 1500 mg/dL or higher. Lp(a) levels are measure using any known method, e.g., an immunological assay, e.g., an enzyme-linked immunosorbent assay (ELISA); a radioimmunoassay (RIA); Western blot analysis; and the like, where antibody specific for apo(a) and/or apoB-100 is used.

In some embodiments, a subject transgenic animal exhibits plasma cholesterol levels greater than about 170 mg/dL, e.g., a plasma cholesterol level of from about 170 mg/dL to about 400 mg/dL, e.g., a plasma cholesterol level of from about 170 mg/dL to about 200 mg/dL, from about 200 mg/dL to about 250 mg/dL, from about 250 mg/dL to about 300 mg/dL, from about 300 mg/dL to about 350 mg/dL, or from about 350 mg/dL to about 400 mg/dL or higher. Plasma cholesterol levels are measured using any known method, including, e.g., the cholesterol oxidase method (Yokode et al. (1990) Science 250:1273-1275); by fluorimetry (Ishibashi et al. (1993) J. Clin. Invest. 92:883-893); a colorimetric assay; and the like.

In some embodiments, a subject transgenic animal exhibits plasma triglyceride levels greater than about 170 mg/dL, e.g., a plasma triglyceride level of from about 170 mg/dL to about 400 mg/dL, e.g., a plasma triglyceride level of from about 170 mg/dL to about 200 mg/dL, from about 200 mg/dL to about 250 mg/dL, from about 250 mg/dL to about 300 mg/dL, from about 300 mg/dL to about 350 mg/dL, or from about 350 mg/dL to about 400 mg/dL or higher. Plasma triglyceride levels are measured using any known method, including, e.g., a calorimetric assay (e.g., Wahlefeld and Bergmeyer (1974) Methods of Enzymatic Analysis 2^{nd} English ed., p. 1831, Academic Press, New York).

In some embodiments, a subject transgenic animal has a plasma level of covalently linked Lp(a) (i.e., apo(a) is covalently linked to apoB-100 in the plasma Lp(a)) that is in excess of 30 mg/dL, e.g., a subject transgenic animal has a plasma level of covalently linked Lp(a) of from about 30 mg/dL to about 1500 mg/dL, e.g., a subject transgenic animal has a plasma level of covalently linked Lp(a) of from about 30 mg/dL to about 40 mg/dL, from about 40 mg/dL to about 50 mg/dL, from about 50 mg/dL to about 100 mg/dL, from about 100 mg/dL to about 150 mg/dL, from about 150 mg/dL to about 200 mg/dL, from about 200 mg/dL to about 250 mg/dL, from about 250 mg/dL to about 300 mg/dL, from about 300 mg/dL to about 350 mg/dL, from about 350 mg/dL to about 400 mg/dL, from about 400 mg/dL to about 450 mg/dL, from about 450 mg/dL to about 500 mg/dL, from about 500 mg/dL to about 550 mg/dL, from about 550 mg/dL to about 600 mg/dL, from about 600 mg/dL to about 650 mg/dL, from about 650 mg/dL to about 700 mg/dL, from about 700 mg/dL to about 750 mg/dL, from about 750 mg/dL to about 800 mg/dL, from about 800 mg/dL to about 900 mg/dL, from about 900 mg/dL to about 1000 mg/dL, from about 1000 mg/dL to about 1100 mg/dL, from about 1100 mg/dL to about 1200 mg/dL, from about 1200 mg/dL to about 1300 mg/dL, from about 1300 mg/dL to about 1400 mg/dL, or from about 1400 mg/dL to about 1500 mg/dL or higher.

In some embodiments, where a subject transgenic animal comprises a transgene encoding apo(a) and apoB-100 is encoded by an endogenous gene (e.g., the animal is not transgenic for apoB-100), the transgene-encoded apo(a) and the endogenously-encoded apoB-100 are covalently linked, e.g., via disulfide bonds. In these embodiments, at least about 20%, at least about 30%, at least about 40%, at least about 50%, at least about 60%, at least about 70%, at least about 80%, or at least about 90%, or more, of the transgene-encoded apo(a) and the endogenously-encoded apoB-100 found in the plasma are covalently linked. In many of these embodiments, the level of apo(a) is from about 30 mg/dL to about 1500 mg/dL. In many of these embodiments, the proportion of the transgene-encoded apo(a) that is covalently linked to the endogenously-encoded apoB-100 ranges from about 20% to about 90% or higher. In many of these embodiments, the plasma level of covalently linked Lp(a) (i.e., Lp(a) that contains apo(a) covalently linked to apoB-100) is greater than 30 mg/dL, e.g., from about 30 mg/dL to about 1500 mg/dL.

In some embodiments, where a subject transgenic animal comprises a transgene encoding apo(a) and a transgene encoding apoB-100, the transgene-encoded apo(a) and the transgene-encoded apoB-100 are covalently linked, e.g., via disulfide bonds. In these embodiments, at least about 20%, at least about 30%, at least about 40%, at least about 50%, at least about 60%, at least about 70%, at least about 80%, or at least about 90%, or more, the transgene-encoded apo(a) and the transgene-encoded apoB-100 found in the plasma are covalently linked. In many of these embodiments, the plasma level of covalently linked Lp(a) (i.e., Lp(a) that contains apo(a) covalently linked to apoB-100) is greater than 30 mg/dL, e.g., from about 30 mg/dL to about 1500 mg/dL.

Whether apo(a) and apoB-100 are covalently linked by a disulfide bond(s) is readily determined using well-known techniques, e.g., by comparing the amount of protein in bands corresponding to apo(a), apoB-100, and Lp(a) in reducing and non-reducing sodium-dodecyl sulfate (SDS)-polyacrylamide gel electrophoresis (PAGE).

In many embodiments, a subject transgenic animal exhibits an atherosclerotic phenotype. An “atherosclerotic phenotype” is characterized by (1) lipid staining lesions in a blood vessel, e.g., the aorta; and/or (2) atherosclerotic plaques comprising Lp(a); and/or (3) stroke; and/or (4) cerebrovascular disease.

In many embodiments, a subject transgenic animal exhibits an increased number of aortic lesions when fed a high fat/high cholesterol diet. For example, in some embodiments, a subject transgenic animal exhibits an increase in aortic lesions of from about 50% to about 2-fold, from about 2-fold to about 4-fold, from about 4-fold to about 5-fold, from about 5-fold to about 10-fold, or from about 10-fold to about 50-fold, compared to a non-transgenic animal of the same species, or compared to an animal transgenic for apo(a) alone, or compared to an animal transgenic for apoB-100 alone.

In some embodiments, a subject transgenic animal develops atherosclerosis when fed a high-fat diet for a period of time. In other embodiments, a subject transgenic animal develops atherosclerosis when fed a low-fat diet. An example of a low-fat diet is a cholate-free mouse-chow diet containing 6% animal fat and <0.04% cholesterol. An example of a high-fat diet is a diet containing 1.25% cholesterol, 7.5% saturated fat as cocoa butter, 7.5% casein and 0.5% sodium cholate.

The transgenic animals of the present invention are other than human, and are typically non-human mammals, including, but not limited to farm animals (pigs, goats, sheep, cows, horses (also known as ungulates or hooved animals, and including ruminants)), rodents (such as mice), and lagomorphs (e.g., rabbits). In some embodiments of interest, a subject transgenic non-human animal is a mouse.

Methods of Making a Subject Transgenic Animal

The invention provides methods of generating a subject transgenic animal. The method generally involves introducing an apo(a) and/or an apoB-100 transgene into a pluripotent or totipotent cell such that the transgene is integrated into the genome of the cell, and transferring the cell into an oviduct of a synchronized recipient female of the same species as the cell.

In some embodiments, a subject transgenic animal is produced by introducing into a single cell embryo a polynucleotide(s) that comprises a nucleotide sequence that encodes an apo(a) and/or an apoB-100 polypeptide, or fragments or variants thereof, in a manner such that the polynucleotide is stably integrated into the DNA of germ line cells of the mature animal, and is inherited in normal Mendelian fashion. In accordance with the invention, a polynucleotide can be introduced into an embryo by a variety of means to produce transgenic animals. For instance, totipotent or pluripotent stem cells, zygotes (fertilized oocytes), embryonic cells, or somatic cells can be transformed by microinjection, calcium phosphate mediated precipitation, liposome fusion, electroporation, retroviral infection or by other means. Where the transformed cell is other than a zygote or embryonic cell, the transformed cells can then be introduced into embryos and incorporated therein to form transgenic animals.

In many embodiments, a polynucleotide is injected into an embryo, e.g., at the single-cell stage, forming a genetically modified embryo, and the genetically modified embryo is allowed to develop into a mature transgenic animal.

In some embodiments, the transgene is introduced into a somatic cell, where the transgene is integrated into the genome, forming a genetically modified somatic cell, and the nucleus of the genetically modified somatic cell is transferred into a single-cell embryo, forming a genetically modified embryo. The genetically modified single-cell embryo is then transferred into an oviduct of a recipient female, and the embryo allowed to develop into a mature transgenic animal.

Any method of making transgenic animals can be used as described, for example, in Transgenic Animal Generation and Use L. M. Houdebine, Harwood Academic Press, 1997; Transgenesis Techniques: Principles and Protocols D. Murphy and D. A. Carter, ed. (June 1993) Humana Press; Transgenic Animal Technology: A Laboratory Handbook C. A. Pinkert, ed. (Jan. 1994) Academic Press; Transgenic Animals F. Grosveld and G Kollias, eds. (July 1992) Academic Press; and Embryonal Stem Cells: Introducing Planned Changes into the Animal Germline M. L. Hooper (January 1993) Gordon & Breach Science Pub; U.S. Pat. No. 6,344,596; U.S. Pat. No. 6,271,436; U.S. Pat. No. 6,218,596; and U.S. Pat. No. 6,204,431; Maga and Murray (1995) Bio/Technol. 13:1452-1457; Ebert et al. (1991) Bio/Technol. 9:835-838; Velander et al. (1992) Proc. Natl. Acad. Sci. USA 89:12003-12007; Wright et al. (1991) Bio/Technol. 9:830-834.

Transgenic animals also can be generated using methods of nuclear transfer or cloning using embryonic or adult cell lines as described for example in Campbell et al. (1996) Nature 380: 64-66; and Wilmut et al. (1997) Nature 385: 810-813. Cytoplasmic injection of DNA can be used, as described in U.S. Pat. No. 5,523,222. Subject transgenic animals can be obtained by introducing a construct comprising apo(a)-encoding and/or an apoB-100-encoding sequences.

Transgenic animals also include somatic transgenic animals, e.g., transgenic animals that include a transgene in somatic cells (and not in germ line cells). Methods of somatic cell transformation are described in the art. See, e.g., Furth et al. (1995) Mol. Biotechnol. 4:121-127.

Methods for making transgenic goats are known in the art. See, e.g., Zou et al. (2002) Mol. Reprod. Dev. 61:164-172; Baldassare et al. (2002) Theriogenol. 57:275-284; and Ko et al. (2000) Transgenic Res. 9:215-222. Methods for making transgenic cows are known in the art, and are described in, e.g., van Berkel et al. (2002) Nat. Biotechnol. 20:484-487. Methods for making transgenic pigs are known in the art. See, e.g., U.S. Pat. Nos. 6,344,596; 6,262,336; and 6,218,596.

Expression Vectors and Transgenes

A subject transgenic animal is typically generated by a method involving introducing into a cell a construct comprising a nucleotide sequence encoding an apo(a) and/or an apoB-100 polypeptide. The present invention provides isolated polynucleotides comprising a subject transgene, and vectors, including expression vectors, comprising the polynucleotides. The present invention further provides host cells, including isolated host cells, e.g., host cells comprising a subject transgene, host cells comprising a subject polynucleotide, and host cells comprising a subject expression vector.

An “apo(a) transgene” includes, at a minimum, a coding region for apolipoprotein(a). In some embodiments, the nucleotide sequence encoding apo(a) is operably linked to a promoter and, optionally, additional control elements, that provide for tissue-specific expression of the transgene in the animal. In other embodiments, the nucleotide sequence encoding apo(a) is not operably linked to any control elements.

Similarly, an “apoB-100 transgene” includes, at a minimum, a coding region for apolipoprotein B-100. In some embodiments, the nucleotide sequence encoding apoB-100 is operably linked to a promoter and, optionally, additional control elements, that provide for tissue-specific expression of the transgene in the animal. In other embodiments, the nucleotide sequence encoding apoB-100 is not operably linked to any control elements.

Apo(a) Transgene

As discussed above, any nucleotide sequence that codes for an apo(a) polypeptide can be used to make a subject transgenic animal, including an apo(a) coding sequence from rat, mouse, human, cow, goat, sheep, chicken, etc., or variant sequences that encode an apo(a) polypeptide. Suitable apo(a) coding sequences include those disclosed in the art. See, e.g., Boonmark et al. (1997) J. Clin. Invest. 100:558-564; GenBank Accession Nos. NM_{—}005577, and X06290.

Sequences that vary from a known coding sequence for a given apo(a) polypeptide can be used, as long as the encoded apo(a) polypeptide has substantially the same activity in promoting an atherosclerotic phenotype. For example, the encoded apo(a) polypeptide can include one or more conservative amino acid substitutions compared to the amino acid sequence of a known apo(a) polypeptide. Non-limiting examples of conservative amino acid substitutions are Phe/Tyr; Ala/Val; Leu/Ile; Arg/His; Ser/Thr; etc. The encoded apo(a) polypeptide can also include insertions or deletions (including truncations) of one or more amino acid residues, compared to the amino acid sequence of a known apo(a) polypeptide. Further, the encoded apo(a) polypeptide can include one or more naturally occurring polymorphisms. The apo(a) polypeptide coding sequence can be completely or partially synthetic. An apo(a) polypeptide coding sequence can also be a consensus sequence, derived, e.g., by comparing the apo(a) polypeptide coding sequences from two or more species, and deriving therefrom a consensus sequence, using standard methods.

Any known coding sequence for an apo(a) polypeptide can be used to make a subject transgenic animal, including an apo(a) polypeptide coding sequence from rat, mouse, human, cow, goat, sheep, etc. The coding sequence can be a cDNA sequence, or a genomic sequence. The coding sequence for the apo(a) polypeptide may be, but need not be, from the same species as the transgenic animal. In certain embodiments, the apo(a) coding sequence is the human apo(a) coding sequence, or a variant thereof.

As discussed apo(a) contains a variable number of copies of a kringle-like domain that has approximately 75% identity with kringle IV of plasminogen. In some embodiments, the apo(a) transgene comprises an apo(a) coding sequence that encodes 17 kringle IV domains. In other embodiments, the apo(a) transgene comprises an apo(a) coding sequence that encodes fewer than 17 kringle IV domains, e.g., the apo(a) transgene comprises a nucleotide sequence that encodes from 1 to 16, from 1 to 3, from 3 to 5, from 5 to 8, from 8 to 10, from 10 to 12, from 12 to 14, or from 14 to 16 kringle IV domains. In some embodiments, an apo(a) transgene comprises a nucleotide sequence that encodes 8 kringle IV domains.

In addition, sequences that vary from a known coding sequence for apo(a) polypeptide can be used, as long as the encoded apo(a) polypeptide has substantially the same activity in contributing to the atherosclerotic phenotype. For example, the encoded apo(a) polypeptide can include one or more conservative amino acid substitutions compared to the amino acid sequence of a known apo(a) polypeptide. Examples of conservative amino acid substitutions are Phe/Tyr; Ala/Val; Leu/Ile; Arg/His; Ser/Thr; etc. The encoded apo(a) polypeptide can also include insertions or deletions (including truncations) of one or more amino acid residues, compared to the amino acid sequence of a known apo(a) polypeptide. Further, the encoded apo(a) polypeptide can include one or more naturally occurring polymorphisms.

A suitable nucleotide sequence encoding an apo(a) polypeptide generally has aa least about 75%, at least about 80%, at least about 85%, at least about 90%, at least about 95%, or at least about 98%, or higher, nucleotide sequence identity with a known coding sequence for apo(a) polypeptide. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings).

Also suitable for use are apo(a) polypeptide coding sequences that hybridize under stringent hybridization conditions to a known apo(a) coding sequence. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. For example, high stringency conditions include aqueous hybridization (e.g., free of formamide) in 6×SSC (where 20×SSC contains 3.0 M NaCl and 0.3 M sodium citrate), 1% sodium dodecyl sulfate (SDS) at 65° C. for about 8 hours (or more), followed by one or more washes in 0.2×SSC, 0.1% SDS at 65° C. For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature.

As noted above, in some embodiments, an apo(a) polypeptide-encoding transgene includes a coding sequence for apo(a) polypeptide operably linked to one or more control sequences, e.g., promoters, 3′ transcriptional control sequences, translational control elements, etc.

In many embodiments, an apo(a) polypeptide transgene includes a coding region for apo(a) polypeptide operably linked to one or more tissue-specific control elements, e.g., a tissue-specific promoter, and optionally additional tissue-specific control elements (e.g., a 3′ untranslated region, an enhancer, and the like). The tissue-specific control element(s) can be heterologous, e.g., not normally operably linked to a apo(a) polypeptide coding sequence in nature, or homologous, e.g., normally operably linked to an apo(a) coding sequence in nature. Tissue-specific control elements provide for expression of the apo(a) polypeptide transgene preferentially in a given tissue, e.g., such control elements are more active (e.g., 2-fold, 5-fold, 10-fold, 20-fold, or 50-fold more active, or greater than 50-fold more active) in a given tissue than in other tissues under normal physiological conditions. A wide variety of tissue-specific promoters are known in the art. In some embodiments, a human apoliprotein E promoter and intron are the control elements. See, e.g., Paik et al. (1988) J Biol Chem. 263(26):13340-9.

Where the control element operably linked to the apo(a) polypeptide coding region in the transgene is an apo(a) polypeptide control element, the apo(a) control element may be altered to provide for increased transcription, increased mRNA stability, and the like, e.g., using random or site-specific mutagenesis techniques. Methods for random and site-specific mutagenesis are well known in the art. Whether a given mutation of a control element increases the level of apo(a) mRNA is readily determined using well-known methods. For example, an expression vector that includes an apo(a) polypeptide promoter operably linked to a reporter gene, e.g., a nucleotide sequence encoding a detectable protein, such as a luciferase-encoding sequence, is introduced into a eukaryotic cell, and the promoter activity is determined by measuring the level of luciferase produced in the cell.

ApoB-100 Transgene

As discussed above, any nucleotide sequence that codes for an apoB-100 polypeptide can be used to make a subject transgenic animal, including an apoB-100 coding sequence from rat, mouse, human, cow, goat, sheep, chicken, etc., or variant sequences that encode an apoB-100 polypeptide. Suitable apoB-100 coding sequences include, e.g., sequences found under GenBank Accession Nos. X04714, M14162, X04505, J02610, M15421, and AH003569.

Sequences that vary from a known coding sequence for a given apoB-100 polypeptide can be used, as long as the encoded apoB-100 polypeptide has substantially the same activity in promoting an atherosclerotic phenotype. For example, the encoded apoB-100 polypeptide can include one or more conservative amino acid substitutions compared to the amino acid sequence of a known apoB-100 polypeptide. Non-limiting examples of conservative amino acid substitutions are Phe/Tyr; Ala/Val; Leu/Ile; Arg/His; Ser/Thr; etc. The encoded apoB-100 polypeptide can also include insertions or deletions (including truncations) of one or more amino acid residues, compared to the amino acid sequence of a known apoB-100 polypeptide. Further, the encoded apoB-100 polypeptide can include one or more naturally occurring polymorphisms. The apoB-100 polypeptide coding sequence can be completely or partially synthetic. An apoB-100 polypeptide coding sequence can also be a consensus sequence, derived, e.g., by comparing the apoB-100 polypeptide coding sequences from two or more species, and deriving therefrom a consensus sequence, using standard methods.

Any known coding sequence for an apoB-100 polypeptide can be used to make a subject transgenic animal, including an apoB-100 polypeptide coding sequence from rat, mouse, human, cow, goat, sheep, etc. The coding sequence can be a cDNA sequence, or a genomic sequence. The coding sequence for the apoB-100 polypeptide may be, but need not be, from the same species as the transgenic animal. In certain embodiments, the apoB-100 coding sequence is the human apoB-100 coding sequence, or a variant thereof.

The nucleotide sequences of mRNAs encoding human apoB-100 polypeptide are known. Exemplary sequences are found under the following GenBank Accession numbers: X04714, M14162, J02610, and M15421.

In addition, sequences that vary from a known coding sequence for apoB-100 polypeptide can be used, as long as the encoded apoB-100 polypeptide has substantially the same activity in contributing to the atherosclerotic phenotype. For example, the encoded apoB-100 polypeptide can include one or more conservative amino acid substitutions compared to the amino acid sequence of a known apo(a) polypeptide. Examples of conservative amino acid substitutions are Phe/Tyr; Ala/Val; Leu/Ile; Arg/His; Ser/Thr; etc. The encoded apoB-100 polypeptide can also include insertions or deletions (including truncations) of one or more amino acid residues, compared to the amino acid sequence of a known apoB-100 polypeptide. Further, the encoded apoB-100 polypeptide can include one or more naturally occurring polymorphisms.

A suitable nucleotide sequence encoding an apoB-100 polypeptide generally has aa least about 75%, at least about 80%, at least about 85%, at least about 90%, at least about 95%, or at least about 98%, or higher, nucleotide sequence identity with a known coding sequence for apoB-100 polypeptide. Sequence similarity is calculated based on a reference sequence, which may be a subset of a larger sequence, such as a conserved motif, coding region, flanking region, etc. A reference sequence will usually be at least about 18 nt long, more usually at least about 30 nt long, and may extend to the complete sequence that is being compared. Algorithms for sequence analysis are known in the art, such as BLAST, described in Altschul et al. (1990), J. Mol. Biol. 215:403-10 (using default settings).

Also suitable for use are apoB-100 polypeptide coding sequences that hybridize under stringent hybridization conditions to a known apoB-100 coding sequence. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. For example, high stringency conditions include aqueous hybridization (e.g., free of formamide) in 6×SSC (where 20×SSC contains 3.0 M NaCl and 0.3 M sodium citrate), 1% sodium dodecyl sulfate (SDS) at 65° C. for about 8 hours (or more), followed by one or more washes in 0.2×SSC, 0.1% SDS at 65° C. For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature.

As noted above, in some embodiments, an apoB-100 polypeptide transgene includes a coding sequence for apoB-100 polypeptide operably linked to one or more control sequences, e.g., promoters, 3′ transcriptional control sequences, translational control elements, etc.

In many embodiments, an apoB-100 polypeptide transgene includes a coding region for apoB-100 polypeptide operably linked to one or more tissue-specific control elements, e.g., a tissue-specific promoter, and optionally additional tissue-specific control elements (e.g., a 3′ untranslated region, an enhancer, and the like). The tissue-specific control element(s) can be heterologous, e.g., not normally operably linked to an apoB-100 polypeptide coding sequence in nature, or homologous, e.g., normally operably linked to an apoB-100 coding sequence in nature. Tissue-specific control elements provide for expression of the apoB-100 polypeptide transgene preferentially in a given tissue, e.g., such control elements are more active (e.g., 2-fold, 5-fold, 10-fold, 20-fold, or 50-fold more active, or greater than 50-fold more active) in a given tissue than in other tissues under normal physiological conditions. A wide variety of tissue-specific promoters are known in the art.

Where the control element operably linked to the apoB-100 polypeptide coding region in the transgene is an apoB-100 polypeptide control element, the apoB-100 control element may be altered to provide for increased transcription, increased mRNA stability, and the like, e.g., using random or site-specific mutagenesis techniques. Methods for random and site-specific mutagenesis are well known in the art. Whether a given mutation of a control element increases the level of apoB-100 mRNA is readily determined using well-known methods. For example, an expression vector that includes an apoB-100 polypeptide promoter operably linked to a reporter gene, e.g., a nucleotide sequence encoding a detectable protein, such as a luciferase-encoding sequence, is introduced into a eukaryotic cell, and the promoter activity is determined by measuring the level of luciferase produced in the cell.

Promoters and Vectors

In general, a transgene (e.g., an apo(a) transgene, an apoB-100 transgene) is operably linked to one or more control elements (“regulatory elements”). Control elements include promoter elements; 3′ control elements; enhancers; introns; elements that confer mRNA stability; and the like.

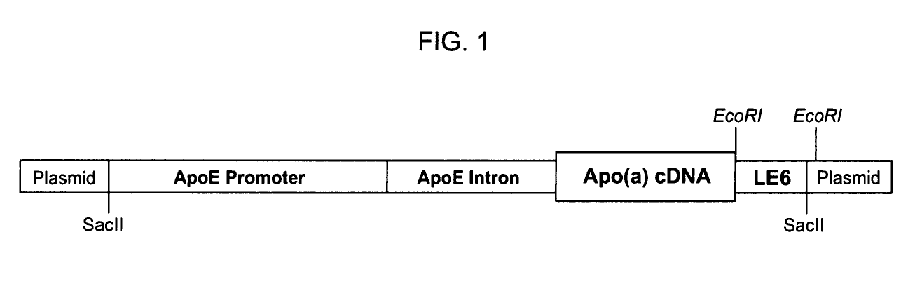

In some embodiments, a transgene is operably linked to one or more control elements that provide for high level expression in the liver. A non-limiting example of a hepatic control region is LE6. Dang et al. (1995) J. Biol. Chem. 270:22577-22585; and Simonet et al. (1993) J. Biol. Chem. 268:8221-8229. A suitable liver cDNA expression vector is described in, e.g., Fan et al. (1994) Proc. Natl. Acad. Sci. USA 91:558-564; and Simonet et al. (1993) J. Biol. Chem. 268:8221-8229. In some embodiments, a subject transgene comprises an apolipoprotein-E (apoE) promoter. In some embodiments, a subject transgene comprises, in order from 5′ to 3′, an apoE promoter, an apoE intron, a nucleotide sequence encoding apo(a), and a 3′ hepatic control region. In some embodiments, the 3′ hepatic control region is LE6, e.g., the 774 base pair nucleotide sequence depicted in FIG. 1 of Dang et al. (1995), supra.

Where the transgenic animal expresses the apo(a) and/or apoB-100 transgene in all tissues, a strong constitutive, or an inducible promoter, is used. Strong constitutive promoters include, but are not limited to, strong promoters that are functional in eukaryotic cells, including a promoter from cytomegalovirus (CMV), mouse mammary tumor virus (MMTV), Rous sarcoma virus (RSV), or adenovirus. Exemplary promoters include the promoter from the immediate early gene of human CMV (Boshart et al., Cell 41:521-530, 1985); the promoter from the long terminal repeat (LTR) of RSV (Gorman et al. (1982 Proc. Natl. Acad. Sci. USA 79:6777-6781); SV40 early promoter; and the adenovirus major late promoter. Typically, a eukaryotic promoter (e.g., a promoter that is functional in a eukaryotic cell) is used.

An apo(a) or apoB-100 transgene is generally provided as part of a vector (e.g., an apo(a) construct; an apoB-100 construct), a wide variety of which are known in the art and need not be elaborated upon herein. Vectors include, but are not limited to, plasmids; cosmids; viral vectors; artificial chromosomes (HACs, YACs, BACs, etc.); mini-chromosomes; and the like. Vectors are amply described in numerous publications well known to those in the art, including, e.g., Short Protocols in Molecular Biology, (1999) F. Ausubel, et al., eds., Wiley & Sons. Vectors may provide for expression of the subject nucleic acids, may provide for propagating the subject nucleic acids, or both.

For expression, e.g., where the transgene includes a promoter, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Where the transgene includes a promoter, an expression vector will generally have convenient restriction sites located near the promoter sequence to provide for the insertion of nucleic acid sequences encoding apo(a) and/or apoB-100. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region.

Utility

A subject transgenic animal is useful in research applications, e.g., to study the atherogenic potential of Lp(a); to study the role of oxidized phospholipids in atherosclerosis; to study the effect of diet on development of atherosclerosis; and the like. A subject transgenic animal is also useful in screening methods to identify agents that treat atherosclerosis.

Research Applications

A subject transgenic animal is useful in research applications, e.g., to study the atherogenic potential of Lp(a); to study the role of oxidized phospholipids in atherosclerosis; to study the effect of diet on development of atherosclerosis; and the like.

Screening Methods

The present invention provides methods of identifying agents that treat atherosclerosis. The methods generally involve administering to a subject transgenic animal a test agent; and determining the effect, if any, of the test agent on an atherosclerotic phenotype.

A test agent of interest is one that reduces an atherosclerotic phenotype by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent.

In some embodiments, a test agent reduces the level of plasma apo(a) by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent.

In some embodiments, a test agent reduces the number and/or size of atherosclerotic plaques or lesions by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the test agent.

As used herein, the term “determining” refers to both quantitative and qualitative determinations and as such, the term “determining” is used interchangeably herein with “assaying,” “measuring,” and the like.

The terms “candidate agent,” “test agent,” “agent”, “substance” and “compound” are used interchangeably herein. Candidate agents encompass numerous chemical classes, typically synthetic, semi-synthetic, or naturally-occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or are readily producible.

Candidate agents may be small organic or inorganic compounds having a molecular weight of more than 50 and less than about 2,500 daltons. Candidate agents may comprise functional groups necessary for structural interaction with proteins, particularly hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In some embodiments, one or more of the following is expressly excluded from the term “candidate agent”: a nucleic acid, an RNA, a double-stranded DNA, a double-stranded RNA, a single-stranded DNA, a single-stranded RNA, an antisense nucleic acid, and an siRNA.

A test agent is administered to a subject transgenic animal, e.g., systemically (e.g., by intravenous injection, by intraperitoneal injection, orally, e.g., by oral gavage, and the like); or locally. The test agent can also be mixed with the diet and/or water, e.g., the test agent is ingested by the animal along with the chow and/or water (ad libitum).

The effect of the test agent is determined by one or more of the following: 1) histological examination of animals; 2) measurement of plasma Lp(a) levels; 3) measurement of plasma cholesterol levels; 4) measurement of plasma triglyceride levels; and 5) measurement of plasma apo(a) levels. Histological examination of animals includes examination of arteries for lipid-staining lesions; presence of apo(a) and/or apoB-100 in arterial lesions; presence of particular lipids in arterial lesions; presence of fatty streaks; and the like. Histological examination can be conducted as described in, e.g., Lawn et al. (1992) Nature 360:670-672; and Sanan et al. (1998) Proc. Natl. Acad. Sci. USA 95:4544-4549.

Levels of plasma apo(a), levels of plasma apoB-100, triglyceride levels, cholesterol levels, and the like can be measured by drawing a blood sample from the animal at various times, e.g., before administering the test agent; and/or at various times following administration of the test agent; and/or at various times during the course of a regimen involving administration of test agent over a period of time.

In some embodiments, a test agent is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), substantially continuously, or continuously, over a period of time ranging from about one day to about one week, from about two weeks to about four weeks, from about one month to about two months, from about two months to about four months, from about four months to about six months, or longer.

In some embodiments, a subject transgenic animal is fed a normal chow diet or a low-fat diet, and a test agent is administered over a period of time of from about 1 day to about 6 months, e.g., for from about 1 day to about 1 week, from about 1 week to about 2 weeks, from about 2 weeks to about 6 weeks, from about 6 weeks to about 8 weeks, from about 8 weeks to about 10 weeks, from about 10 weeks to about 12 weeks, from about 12 weeks to about 4 months, from about 4 months to about 5 months, or from about 5 months to about 6 months or longer.

In other embodiments, a subject transgenic animal is fed a high-fat diet, and a test agent is administered over a period of time of from about 3 days to about 6 months, e.g., for from about 3 days to about 1 week, from about 1 week to about 2 weeks, from about 2 weeks to about 6 weeks, from about 6 weeks to about 8 weeks, from about 8 weeks to about 10 weeks, from about 10 weeks to about 12 weeks, from about 12 weeks to about 4 months, from about 4 months to about 5 months, or from about 5 months to about 6 months or longer.

Agents

The present invention provides agents identified by a subject screening method; and compositions, including pharmaceutical compositions, comprising the agents.

In many embodiments, the agent is a small molecule, e.g., a small organic or inorganic compound having a molecular weight of more than 50 and less than about 2,500 daltons. Agents may comprise functional groups necessary for structural interaction with proteins, particularly hydrogen bonding, and may include at least an amine, carbonyl, hydroxyl or carboxyl group, and may contain at least two of the functional chemical groups. The agents may comprise cyclical carbon or heterocyclic structures and/or aromatic or polyaromatic structures substituted with one or more of the above functional groups. Agents are also found among biomolecules including peptides, saccharides, fatty acids, steroids, purines, pyrimidines, derivatives, structural analogs or combinations thereof.

In some embodiments, an active agent is a peptide. Suitable peptides include peptides of from about 3 amino acids to about 50, from about 5 to about 30, or from about 10 to about 25 amino acids in length. In some embodiments, a peptide exhibits one or more of the following activities: inhibits binding of apo(a) to apoB-100; inhibits incorporation of Lp(a) into particles; inhibits formation of a disulfide bond between apo(a) and apoB-100.

Peptides can include naturally-occurring and non-naturally occurring amino acids. Peptides may comprise D-amino acids, a combination of D- and L-amino acids, and various “designer” amino acids (e.g., β-methyl amino acids, Cα-methyl amino acids, and Nα-methyl amino acids, etc.) to convey special properties to peptides. Additionally, peptide may be a cyclic peptide. Peptides may include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carboline (D and L); HIC (histidine isoquinoline carboxylic acid); and HIC (histidine cyclic urea). Amino acid analogs and peptidomimetics may be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analog; amide bond isostere; tretrazol; and the like.

A peptide may be a depsipeptide, which may be a linear or a cyclic depsipeptide. Kuisle et al. (1999) Tet. Letters 40:1203-1206. “Depsipeptides” are compounds containing a sequence of at least two alpha-amino acids and at least one alpha-hydroxy carboxylic acid, which are bound through at least one normal peptide link and ester links, derived from the hydroxy carboxylic acids, where “linear depsipeptides” may comprise rings formed through S-S bridges, or through an hydroxy or a mercapto group of an hydroxy-, or mercapto-amino acid and the carboxyl group of another amino- or hydroxy-acid but do not comprise rings formed only through peptide or ester links derived from hydroxy carboxylic acids. “Cyclic depsipeptides” are peptides containing at least one ring formed only through peptide or ester links, derived from hydroxy carboxylic acids.

Peptides may be cyclic or bicyclic. For example, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art

The term “bicyclic” refers to a peptide in which there exists two ring closures. The ring closures are formed by covalent linkages between amino acids in the peptide. A covalent linkage between two nonadjacent amino acids constitutes a ring closure, as does a second covalent linkage between a pair of adjacent amino acids which are already linked by a covalent peptide linkage. The covalent linkages forming the ring closures may be amide linkages, i.e., the linkage formed between a free amino on one amino acid and a free carboxyl of a second amino acid, or linkages formed between the side chains or “R” groups of amino acids in the peptides. Thus, bicyclic peptides may be “true” bicyclic peptides, i.e., peptides cyclized by the formation of a peptide bond between the N-terminus and the C-terminus of the peptide, or they may be “depsi-bicyclic” peptides, i.e., peptides in which the terminal amino acids are covalently linked through their side chain moieties.

A desamino or descarboxy residue can be incorporated at the terminii of the peptide, so that there is no terminal amino or carboxyl group, to decrease susceptibility to proteases or to restrict the conformation of the peptide. C-terminal functional groups include amide, amide lower alkyl, amide di(lower alkyl), lower alkoxy, hydroxy, and carboxy, and the lower ester derivatives thereof, and the pharmaceutically acceptable salts thereof.

In addition to the foregoing N-terminal and C-terminal modifications, a peptide or peptidomimetic can be modified with or covalently coupled to one or more of a variety of hydrophilic polymers to increase solubility and circulation half-life of the peptide. Suitable nonproteinaceous hydrophilic polymers for coupling to a peptide include, but are not limited to, polyalkylethers as exemplified by polyethylene glycol and polypropylene glycol, polylactic acid, polyglycolic acid, polyoxyalkenes, polyvinylalcohol, polyvinylpyrrolidone, cellulose and cellulose derivatives, dextran and dextran derivatives, etc. Generally, such hydrophilic polymers have an average molecular weight ranging from about 500 to about 100,000 daltons, from about 2,000 to about 40,000 daltons, or from about 5,000 to about 20,000 daltons. The peptide can be derivatized with or coupled to such polymers using any of the methods set forth in Zallipsky, S., Bioconjugate Chem., 6:150-165 (1995); Monfardini, C, et al., Bioconjugate Chem., 6:62-69 (1995); U.S. Pat. Nos. 4,640,835; 4,496,689; 4,301,144; 4,670,417; 4,791,192; 4,179,337 or WO 95/34326.

Other agents include peptide aptamers. Peptide aptamers are peptides or small polypeptides that act as dominant inhibitors of protein function. Peptide aptamers specifically bind to target proteins, blocking their function ability. Kolonin and Finley, PNAS (1998) 95:14266-14271. Due to the highly selective nature of peptide aptamers, they may be used not only to target a specific protein, but also to target specific functions of a given protein (e.g. a signaling function). Further, peptide aptamers may be expressed in a controlled fashion by use of promoters which regulate expression in a temporal, spatial or inducible manner. Peptide aptamers act dominantly; therefore, they can be used to analyze proteins for which loss-of-function mutants are not available.

Peptide aptamers that bind with high affinity and specificity to a target protein may be isolated by a variety of techniques known in the art. Peptide aptamers can be isolated from random peptide libraries by yeast two-hybrid screens (Xu et al., PNAS (1997) 94:12473-12478). They can also be isolated from phage libraries (Hoogenboom et al., Immunotechnology (1998) 4:1-20) or chemically generated peptides/libraries.

Intracellularly expressed antibodies, or intrabodies, are single-chain antibody molecules designed to specifically bind and inactivate target molecules inside cells. Intrabodies have been used in cell assays and in whole organisms. Chen et al., Hum. Gen. Ther. (1994) 5:595-601; Hassanzadeh et al., Febs Lett. (1998) 16(1, 2):75-80 and 81-86. Inducible expression vectors can be constructed with intrabodies that react specifically with Lp(a), apo(a), or apoB-100 protein. These vectors can be introduced into model organisms and studied in the same manner as described above for aptamers.

In some of the invention, the active agent is an agent that modulates, and generally decreases or down regulates, the expression of the gene encoding apo(a) and/or apoB-100 in the host. Such agents include, but are not limited to, antisense RNA, interfering RNA, ribozymes, and the like.

In some embodiments, the active agent is an interfering RNA (RNAi). RNAi includes double-stranded RNA interference (dsRNAi). Use of RNAi to reduce a level of a particular mRNA and/or protein is based on the interfering properties of double-stranded RNA derived from the coding regions of gene. In one example of this method, complementary sense and antisense RNAs derived from a substantial portion of the apo(a) gene are synthesized in vitro. The resulting sense and antisense RNAs are annealed in an injection buffer, and the double-stranded RNA injected or otherwise introduced into the subject (such as in their food or by soaking in the buffer containing the RNA). See, e.g., WO99/32619. In another embodiment, dsRNA derived from an apo(a) gene is generated in vivo by simultaneous expression of both sense and antisense RNA from appropriately positioned promoters operably linked to apo(a) coding sequences in both sense and antisense orientations.

Antisense molecules can be used to down-regulate expression of the gene encoding apo(a) in cells. Antisense compounds include ribozymes, external guide sequence (EGS) oligonucleotides (oligozymes), and other short catalytic RNAs or catalytic oligonucleotides which hybridize to the target nucleic acid and modulate its expression.

The anti-sense reagent may be antisense oligonucleotides (ODN), particularly synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules may be administered, where a combination may comprise multiple different sequences.

Antisense molecules may be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression (see Wagner et al. (1996), Nature Biotechnol. 14:840-844).

A specific region or regions of the endogenous sense strand mRNA sequence is chosen to be complemented by the antisense sequence. Selection of a specific sequence for the oligonucleotide may use an empirical method, where several candidate sequences are assayed for inhibition of expression of the target gene in an in vitro or animal model. A combination of sequences may also be used, where several regions of the mRNA sequence are selected for antisense complementation.

Antisense oligonucleotides may be chemically synthesized by methods known in the art (see Wagner et al. (1993), supra, and Milligan et al., supra.) Preferred oligonucleotides are chemically modified from the native phosphodiester structure, in order to increase their intracellular stability and binding affinity. A number of such modifications have been described in the literature, which modifications alter the chemistry of the backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage. Sugar modifications are also used to enhance stability and affinity. The β-anomer of deoxyribose may be used, where the base is inverted with respect to the natural α-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5- propynyl-2′-deoxyuridine and 5-propynyl-2′-deoxycytidine have been shown to increase affinity and biological activity when substituted for deoxythymidine and deoxycytidine, respectively.

Exemplary modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Oligonucleotides having a morpholino backbone structure (Summerton, J. E. and Weller D. D., U.S. Pat. No. 5,034,506) or a peptide nucleic acid (PNA) backbone (P. E. Nielson, M. Egholm, R. H. Berg, O. Buchardt, Science 1991, 254: 1497) can also be used. Morpholino antisense oligonucleotides are amply described in the literature. See, e.g., Partridge et al. (1996) Antisense Nucl. Acid Drug Dev. 6:169-175; and Summerton (1999) Biochem. Biophys. Acta 1489:141-158.

As an alternative to anti-sense inhibitors, catalytic nucleic acid compounds, e.g. ribozymes, anti-sense conjugates, etc. may be used to inhibit gene expression. Ribozymes may be synthesized in vitro and administered to the patient, or may be encoded on an expression vector, from which the ribozyme is synthesized in the targeted cell (for example, see International patent application WO 9523225, and Beigelman et al. (1995), Nucl. Acids Res. 23:4434-42). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al. (1995), Appl. Biochem. Biotechnol. 54:43-56.

Formulations, Dosages, and Routes of Administration

The invention provides formulations, including pharmaceutical formulations, comprising a subject agent that treats atherosclerosis. In general, a formulation comprises an effective amount of an agent that treats atherosclerosis. An “effective amount” means a dosage sufficient to produce a desired result, e.g., a reduction in atherosclerotic plaques; a reduction in atherosclerotic plaque formation; etc, as compared to a control.

Formulations

In the subject methods, the active agent(s) may be administered to the host using any convenient means capable of resulting in the desired reduction atherosclerosis. Thus, the agent can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Other modes of administration will also find use with the subject invention. For instance, an agent of the invention can be formulated in suppositories and, in some cases, aerosol and intranasal compositions. For suppositories, the vehicle composition will include traditional binders and carriers such as, polyalkylene glycols, or triglycerides. Such suppositories may be formed from mixtures containing the active ingredient in the range of about 0.5% to about 10% (w/w), preferably about 1% to about 2%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the mucosa.

An agent of the invention can be administered as injectables. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, eg., Remington's Pharmaceutical Sciences, Mack Publishing Company, Easton, Pa., 17th edition, 1985. The composition or formulation to be administered will, in any event, contain a quantity of the agent adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Dosages

Although the dosage used will vary depending on the clinical goals to be achieved, a suitable dosage range is one which provides up to about 1 μg to about 1,000 μg or about 10,000 μg of a subject agent can be administered in a single dose. Alternatively, a target dosage of an agent that treats atherosclerosis can be considered to be about in the range of about 0.1-1000 μM, about 0.5-500 μM, about 1-100 μM, or about 5-50 μM in a sample of host blood drawn within the first 24-48 hours after administration of the agent.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

Routes of administration

A subject agent is administered to an individual using any available method and route suitable for drug delivery, including in vivo and ex vivo methods, as well as systemic and localized routes of administration.

Conventional and pharmaceutically acceptable routes of administration include oral, intranasal, intramuscular, subcutaneous, intradermal, topical application, intravenous, rectal, nasal, and other, parenteral, routes of administration. Routes of administration may be combined, if desired, or adjusted depending upon the agent and/or the desired effect. The composition can be administered in a single dose or in multiple doses.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The agent can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts (wherein the term “host” is used interchangeably herein with the terms “subject” and “patient”) are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits with unit doses of the active agent, e.g. in oral or injectable doses, are provided. In such kits, in addition to the containers containing the unit doses will be an informational package insert describing the use and attendant benefits of the drugs in treating pathological condition of interest. Preferred compounds and unit doses are those described herein above.

Treatment Methods

The present invention provides methods of treating atherosclerosis, the methods generally involving administering to an individual in need thereof an effective amount of a subject agent.

Subjects Amenable to Treatment

Subjects amenable to treatment using the methods and agents described herein include individuals who are at risk of developing atherosclerosis, where such risk is determined on the basis of, e.g., genetic testing, diet, etc.; and individuals who are known to have atherosclerotic plaques.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s, second(s); min, minute(s); hr, hour(s); and the like.

### Example 1

### Generation and Characterization of Lp(a) Transgenic Animals

Experimental Procedures

Generation of Apo(a) Transgenic Mice

Wild-type human apo(a) cDNA encoded the following kringles: IV-1, IV-2, a fusion of IV-3 and IV-5, IV-6 to IV-10, V, and the protease domain as described (8). The vector [pRK5ha8] was digested with XhoI, polished with cloned Pfu DNA polymerase (Stratagene, La Jolla, Calif.), and cut with EcoRI. The apo(a) fragment was inserted into a liver cDNA expression vector (9, 10) that had been digested with KpnI, polished as described above, and cut with EcoRI. Using primers 5′-CGGGAATTCTGCAGGCTCAGAG3′ (SEQ ID NO:01) and 5′-GGGAATTCGAGCTCCGCGGCAGCCTGACCA3′ (SEQ ID NO:02), a PCR product of the apoE hepatic control region (LE6) was generated to introduce SacII and EcoRI sites, and the LE6 was fused to the 3′ end of the apo(a) cDNA. The transgenic mice were created from an 8.6-kb fragment consisting of the apoE promoter, apoE intron, apo(a) cDNA, and LE6 that was linearized with SacII, purified, and microinjected into embryos of C57/BL6× SJL (50/50) mice. Founders generated with the apo(a) construct [pLIVha8] were identified by PCR.

Breeding of Apo(a) and Lp(a) Mice

Hemizygous mice expressing human apo(a) were crossed with hemizygous mice expressing human apoB-100 (11, 12). The mice expressing human apoB-100, but not human apoB-48, were generated with the apoB-100 “Leu-Leu” plasmid to increase the yield of apoB-100. It contains a CAA to CTA mutation in codon 2153 that effectively abolishes the formation of apoB-48, which is formed by an editing mechanism in mouse liver (11). All mice were on a C57/BL6× SJL (50/50) background, weaned at 28 days of age, housed in a barrier facility with a 12-h light/12-h dark cycle, and fed a chow diet containing 4.5% fat (Ralston Purina, St. Louis, Mo.).

Northern Blot Analysis

mRNA was isolated from several tissues and organs from one transgenic offspring of founder 23 and a total of 10 μg was electrophoresed in a 1% agarose gel containing 18% formaldehyde. In addition, liver mRNA from the transgenic mouse (10.0, 7.5, 5.0, and 2.5 μg) and from a nontransgenic littermate (10 μg) were subjected to electrophoresis. After transfer to nylon membrane (Schleicher & Schuell, Keene, N.H.), the mRNA was hybridized to an apo(a) cDNA probe labeled with [³²P]dCTP in Quickhyb solution (Stratagene, La Jolla, Calif.) at 65° C. for 2 h. The blot was washed in 2× standard saline citrate (150 mmol/l NaCl, 15 mmol/l sodium citrate) and 0.1% SDS at 55° C. for 30 min, in 0.1× standard saline citrate and 0.1% SDS at 60° C. for 1 h, and exposed to x-ray film overnight. Signals were quantified with a phosphorimager and quantification software (Bio-Rad Quantity One, Philadelphia, Pa.).

Detection of apo(a) and Lp(a)

Plasma concentrations of human apo(a) and Lp(a) were estimated by SDS-PAGE and Western blotting of mouse plasma with rabbit polyclonal antibodies against human apo(a) (Cortex, San Leandro, Calif.) and human apoB (HB2). Mouse plasma and control human plasma (apo(a) ˜500 kDa) were subjected to SDS-PAGE with 5% gels under reducing and nonreducing conditions, transferred to nitrocellulose, and incubated first with the primary antibodies (1:5,000 for antihuman apo(a) and 1:15,000 for antihuman apoB) and then with horseradish peroxidase-conjugated anti-rabbit antibody (Amersham, Little Chalfont, United Kingdom). Signals were generated by incubating the membranes with chemiluminescent reagent (Amersham) and exposing them to x-ray film (Fuji, Tokyo, Japan) and were quantified with a phosphorimager as described above. Apo(a) and Lp(a) protein plasma concentrations were also determined with a direct-binding, double monoclonal antibody-based enzyme-linked immunoassay (ELISA) as described (13). The capture monoclonal antibody (a-6) is directed to an epitope in apo(a) kringle IV type 2, and the detection antibody (a-40) is directed to a unique epitope in kringle IV type 9. Because each apo(a) molecule contains only one kringle IV type 9, this assay is insensitive to apo(a) isoform size heterogeneity; the results are expressed in nmol/l. This assay was used to quantitate apo(a) levels in plasma of transgenic mice expressing either human apo(a) or both human apo(a) and human apoB.

Formation of Lp(a) in vitro

Disulfide linkage formation between apo(a) and apoB in vitro was assessed as described (14). Different dilutions of plasma from human apo(a) transgenic mice (diluted in 150 mmol/l NaCl) were incubated with 4 μl of plasma from human apoB-100 transgenic mice for 5 min at 37 ° C. The formation of Lp(a) was determined in these mixtures by nonreduced SDS-PAGE and antibodies against human apo(a) and human apoB-100. Lp(a) was identified by apoB-100 of Lp(a) that migrates at a higher molecular weight than apoB-100 of free LDL.

Lipid and Lipoprotein Determination

Tail blood from 8-14 weeks-old mice that had been fasted for 4 h was centrifuged at 16,000 g for 10 min at 4° C. to isolate the plasma. Lipoprotein electrophoresis of pooled plasma was performed with 1% agarose gels and 1.3% barbital buffer. The gels were dried and stained with Fat Red 7B (Helena Laboratories, Beaumont, Tex.) to identify lipoprotein bands. To determine the lipoprotein profiles, 120 μl of mouse plasma was size-fractionated by fast-performance liquid chromatography (FPLC) on a Superose 6 column (Amersham) that had been equilibrated with phosphate-buffered saline (PBS) containing 1 mmol/l EDTA. The distribution of apo(a) and Lp(a) was determined by Western blot analysis as described above. Cholesterol and triglyceride levels in plasma and FPLC fractions were determined with colorimetric assays (Abbott, Abbott Park, Ill. (standards) and Roche, Mannheim, Germany (reagents)).

Density Gradient Ultracentrifugation

Density gradient ultracentrifugation was carried out as described (15). Briefly, a nonlinear salt gradient was constructed to maximize the separation of LDL, Lp(a), and high density lipoprotein (HDL) classes. Very low density lipoprotein (VLDL) remained at the top of the tube. The gradient consisted of 2 ml of 1.21 g/ml NaCl, 3 ml of 4 mol/l NaCl, a mixture of 0.5 ml of mouse plasma and 0.5 ml of 150 mmol/l NaCl. The rest of the tube was filled to a total of 13.2 ml with 670 mmol/l NaCl and this discontinuous gradient was centrifuged in a swinging bucket rotor SW41 (Beckman, Fullerton, Calif.) at 35,000 rpm for 64 h at 15° C. At the end of the run, the tube was pierced at the bottom and 20 660-μl fractions were collected. The density of each fraction was calculated from the refractive index (Bausch & Lomb, Rochester, N.Y.), and Lp(a) was determined by Western blot analysis as described above.

Analysis of Oxidized Phospholipid Content

The oxidized phospholipid content of human or murine apoB-100 particles in mouse plasma was determined by modifications of a chemiluminescent immunoassay as described (16, 17). This sandwich assay uses monoclonal antibody MB47 to capture human apoB-100 from murine plasma and a biotin-labeled monoclonal antibody (EO6) to detect oxidized phospholipids bound to the captured apoB-100 particles. MB47 is specific for human apoB-100 and does not bind to murine apoB-100 (18). EO6, an IgM autoantibody cloned from apoE-deficient mice, binds to oxidized LDL (O×LDL), and specifically to oxidized phospholipid containing phosphorylcholine (PC); it does not bind to PC-containing phospholipids that are not oxidized (19, 20). MB47 (5 μg/ml) was added to 96-well microtiter plates (Microlite2, Dynex Technologies, Chantilly, Va.) in 50 nmol/l Tris-HCL (ph 7.4) containing 150 nmol/l NaCl, 0.27 mmol/l EDTA, and 0.02% NaN₃ Tris-buffered saline (TBS) and incubated overnight at 4° C. The plates were washed three times with TBS washing buffer containing apoprotinin (0.001%) in an automated plate washer, and 1% bovine serum albumin (BSA) in PBS was added to all wells for 45 min at room temperature to block nonspecific binding. The plates were then washed and 50 μl of the murine plasma diluted 1:100 in PBS containing 1% BSA was added for 1 h at room temperature. Preliminary experiments showed that a 1:100 dilution of the mouse plasma containing the human apoB-100 did not saturate the bound MB47. To determine the relative amounts of human apoB-100 bound by MB47 in individual wells, biotin-labeled goat anti-human apoB-100 (Biodesign International, Kennebunk, ME) was added to each well for 1 h at room temperature. The plates were washed with TBS and incubated with 10,000-fold diluted alkaline phosphatase-labeled NeutrAvidin (Pierce, Rockford, Ill.) in TBS buffer containing 1% BSA, 1 mM MgCl₂, and 1 mM ZnCl₂ for 1 h at room temperature. The plates were then washed four times with washing buffer and incubated with 50% Lumi-Phos 530 in distilled water (25 μl/well) for 1.5 h at room temperature in the dark. The chemiluminescence was read on a MLX microtiter plate luminometer (Dynex Technologies). Data are expressed in relative light units (RLU), measured over 100 msec.

To detect endogenous murine apoB-100, the same procedure was utilized except that a monoclonal antibody (LF3, 5 μg/ml) specific for murine apoB-100 (21) was used to capture murine apoB-100, and the biotin-labeled monoclonal antibody LF5 (21) (1 μg/ml) was used for detection. LF5 was biotinylated with EZ-Link Sulfo-NHS-Biotin (Pierce). Preliminary experiments confirmed that LF3 and LF5 did not bind to human apoB-100.

Determination of Oxidized Phospholipid Epitopes Present on Human or Murine ApoB-100

PC-containing oxidized phospholipid epitopes on the captured human and murine apoB-100 were detected with monoclonal antibody EO6 (9). Biotinylated EO6 (conjugated with EZ-Link Biotin-LC-Hydrazide, Pierce) was added to microtiter wells containing captured human or murine apoB-100 particles at 1.5 (human) or 50 μl/well (murine) in 1% BSA/TBS buffer. The amount of EO6 bound was then determined using the chemiluminescent technique described above and expressed in RLU. The amount of EO6 epitopes (oxidized phospholipids) bound per apoB-100 was then determined by dividing the bound EO6 RLU by the apoB-100 RLU in parallel wells to yield a ratio of EO6/apoB-100. All samples were measured in a single assay; the intra-assay coefficient of variation was 8-10%.

Determination of Apo(a) on Human or Murine apoB-100

Human or murine apoB-100 was captured with MB47 or LF3, respectively, as described above. Monoclonal antibody LPA4, which is specific for apo(a) and does not cross-react with plasminogen, was biotinylated using EZ-Link Sulfo-NHS-Biotin and added to each well containing captured apoB-100 at a concentration of 0.5 μg/ml in 1% BSA/TBS buffer, and then detected with the chemiluminescence technique described above.

Statistical Analysis

Statistical analysis was done by one-way analysis of variance (ANOVA) using MicroCal Origin version 6.1 (MicroCal Software, Northampton, Mass.),

Results

Generation of Apo(a) Mice

Mice expressing human apo(a) were generated with an apo(a) cDNA containing eight plasminogen kringle IV-like domains followed by a kringle V-like domain and the protease-like domain (8). This apo(a) cDNA encodes most variants of the kringle IV-like domain and represents a small isoform that is more likely to be associated with high Lp(a) plasma concentrations in the human population (22, 23). The human cDNA was inserted into a liver expression vector containing 3.9 kb of 5′-flanking promoter and 0.77 kb of 3′-flanking hepatic control region (LE6), both from the apoE gene (FIG. 1).

FIG. 1. Construct for liver-specific expression of human apo(a). Construct used to generate apo(a) transgenic mice is illustrated in the 5′ to 3′ orientation. Expression of apo(a) was driven by a 3.0-kb apoE promoter, a 0.9-kb apoE intron and the 0.77-kb hepatic control region (LE6) located downstream of the apo(a) gene. The construct consisted of a SacII—SacII fragment of 8.6 kb was microinjected into mouse embryos.

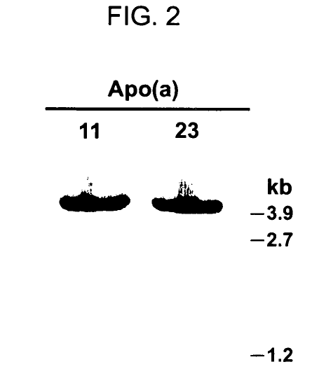

To create transgenic mice, a purified 8.6-kb fragment of the vector that contained apo(a) cDNA was microinjected into mouse embryos. Genomic DNA of each human apo(a) founder mouse was identified by PCR (FIG. 2).

FIG. 2. Generation of transgenic mice expressing human apo(a). Genomic DNA from apo(a) transgenic mice was amplified by PCR, digested with NotI, and analyzed by agarose gels. Apo(a)-expressing mice (11 and 23) were identified by a single DNA fragment of ˜3.9 kb.

Characterization of Apo(a) Mice

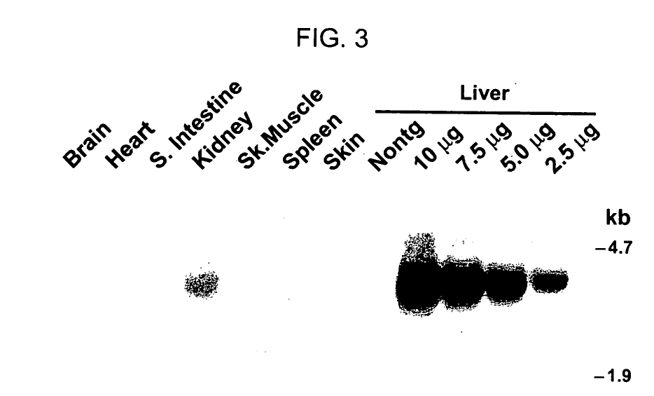

To determine the tissue-specific expression pattern of apo(a), northern blot analysis was performed with tissue from one transgenic and one nontransgenic offspring of founder 23. As expected, apo(a) mRNA (˜3.9 kb) was detected predominantly in the liver of the transgenic mice (FIG. 3).

FIG. 3. Northern blot demonstrating the tissue specificity of apo(a) mRNA expression. Total RNA from various tissues of one transgenic offspring of founder 23 and total liver RNA of a nontransgenic (Nontg) littermate mouse were examined for the presence of apo(a) mRNA. Total RNA (10 μg) was used in all cases except for the liver from the transgenic mouse, where increasing amounts (2.5, 5.0, 7.5 and 10 μg) were used. A ³²P-labeled human apo(a) cDNA was used as a probe.

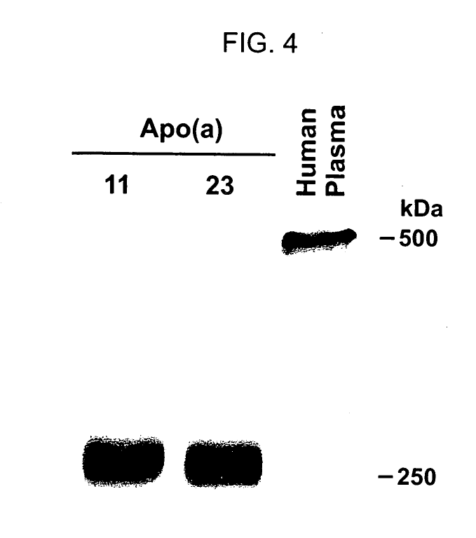

Low levels of expression were observed in the kidney and the brain. Plasma samples from selected lines of transgenic apo(a) mice were tested for protein expression by SDS-PAGE and immunoblot analysis (FIG. 4).

FIG. 4. Relative levels of apo(a) in several lines of transgenic mice expressing human apo(a). Apo(a) in plasma (1 μl) from mice expressing apo(a) (11 and 23) and a human sample was resolved by SDS-PAGE and apo(a) was detected by Western blotting. Apo(a) in plasma (1 μl) from a human subject expressing a high molecular weight form of apo(a) is shown as a positive control.

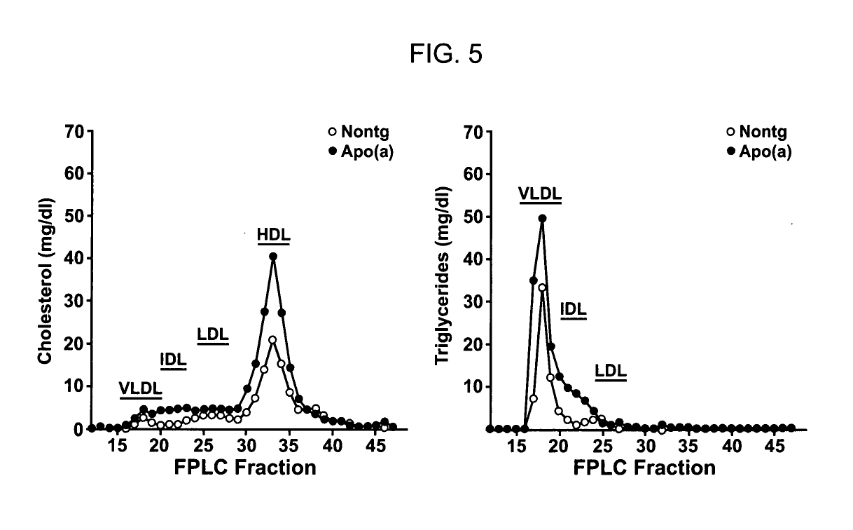

The apo(a) in the human plasma control showed a high molecular weight isoform, whereas the mouse plasma contained a small apo(a) isoform (˜250 kDa) as expected. The plasma cholesterol level was 112±5 mg/dl in apo(a)-expressing mice (n=7) and 70±4 mg/dl in nontransgenic littermates (n=6) (p<0.0001). Their triglyceride levels were not significantly different (71±11 mg/dl versus 45±6 mg/dl, respectively). The distribution of lipids within the various lipoprotein fractions of these mice on a chow diet was assessed after FPLC fractionation. Most of the cholesterol was associated with HDL in both groups (FIG. 5). However, the transgenic mice had higher HDL levels than the controls. Triglycerides were found predominantly in the VLDL in nontransgenic mice and in intermediate density lipoprotein (IDL)-sized particles in the apo(a)-expressing mice.

FIG. 5. Lipoprotein profiles of nontransgenic (“nontg”) and apo(a) transgenic mice. Pooled plasma from nontransgenic (n=6) or apo(a) expressing (n=7) mice was size-fractionated by FPLC and the cholesterol and triglyceride content of-each fraction was measured.

Formation of Lp(a) in vitro

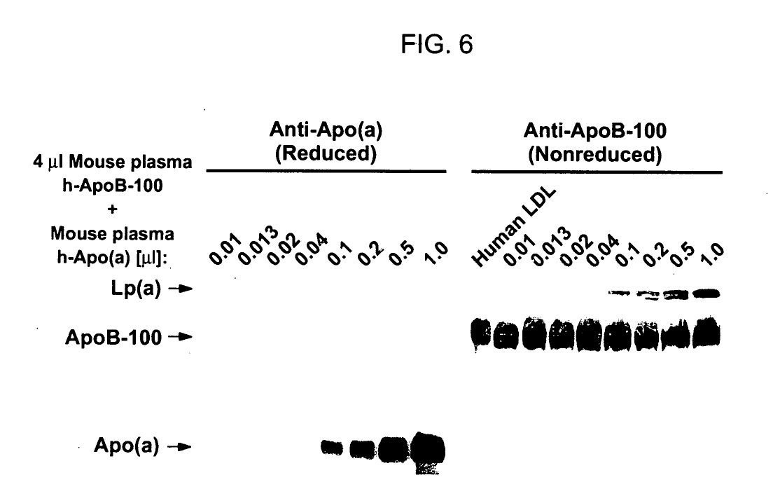

To examine the ability of apo(a) to bind covalently to human apoB-100 and form Lp(a), increasing amounts of plasma from an apo(a)-expressing mouse were incubated with plasma from a mouse expressing human apoB-100, separated by SDS-PAGE, and subjected to immunoblot analysis with antibodies against apo(a) or apoB-100. In the reduced gel, increasing levels of apo(a) were detected in the immunoblot (FIG. 6, left). In the nonreduced gel, nonbound apoB-100 was observed at 500 kDa, while the formation of Lp(a) was evident by the appearance of apoB-100 covalently bound to apo(a) in the higher molecular weight band (FIG. 6, right). Increased levels of apo(a) led to increased formation of Lp(a).

FIG. 6. Apo(a)-dependent formation of Lp(a) in vitro. Increasing concentrations of apo(a) transgenic mouse plasma were mixed with human apoB-100 transgenic mouse plasma (4 μl). After a 5-min incubation at 37° C., apo(a) and Lp(a) in the mixtures was quantified by immunoblot analysis after SDS-PAGE under reducing (left) and nonreducing (right) conditions, respectively. Formation of the disulfide linkage between apo(a) and apoB-100 was evident on a nonreduced gel with increasing concentrations of apo(a) because the larger Lp(a) complex migrates slower than the LDL as detected with the antibody against human apoB.

Generation of Mice Expressing Lp(a) in vivo

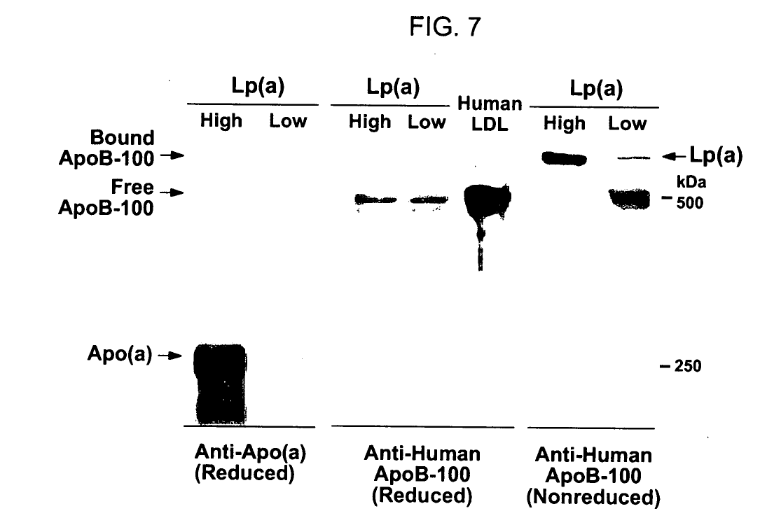

To generate mice expressing Lp(a) in vivo, we bred mice expressing high or low levels of apo(a) with transgenic mice that were hemizygous for human apoB-100 expression. Offspring that carried both transgenes expressed high or low levels of Lp(a). As shown by immunoblotting of mouse plasma separated by SDS-PAGE under reducing and nonreducing conditions, high- and low-expressing mice (FIG. 7, left panel) expressed similar amounts of apoB-100 (FIG. 7, middle panel). In the high expresser, most of the apoB-100 was covalently bound to apo(a) (FIG. 7, right panel). In the low expresser, apo(a) was limiting and therefore only a small amount of apoB-100 was assembled into Lp(a) (FIG. 7, right panel). The high molecular weight Lp(a) band also contained apo(a) as detected by immunoblotting for apo(a).

FIG. 7. Formation of Lp(a) in vivo in mice expressing high or low levels of apo(a) together with human apoB-100. Mice expressing high or low levels of apo(a) were crossed with transgenic mice expressing human LDL. Plasma proteins from transgenic mice expressing both human apo(a) and human apoB-100 were subjected to SDS-PAGE with 5% gels under reducing and nonreducing conditions, transferred to nitrocellulose, and immunoblotted with antibodies against human apo(a) and human apoB. The expression of apo(a) at high levels (left) resulted in formation of high levels of Lp(a) (right) and most of the apoB-100 is bound to apo(a). Low-level expression of apo(a) (left) resulted in low levels of Lp(a) (right) as indicated by the amount of free and bound apoB-100 in both mice. High- and low-expressing mice expressed similar amounts of apoB-100 (middle).

Characterization of Lp(a) High-Expresser Mice

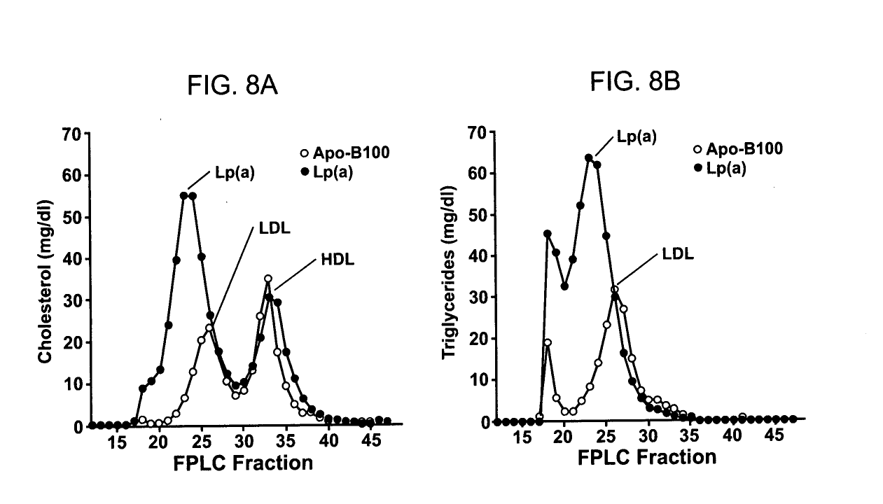

The Lp(a) high-expresser mice that were hemizygous for apo(a) and hemizygous for apoB-100 had Lp(a) mass plasma levels of 701±59 mg/dl (1,928±162 nmol/l). Total plasma cholesterol levels were higher in the Lp(a) mice than in the apoB-100-expressing mice (246±22 mg/dl (n=3) versus 169±6 mg/dl (n=8), p<0.001), as were the plasma triglycerides (281±66 versus 174±11 mg/dl, p<0.05). As shown by density gradient ultracentrifugation, the mean density of Lp(a) was 1.075 g/ml. The lipoprotein profiles of Lp(a) and apoB-100 mice on a chow diet were determined after FPLC fractionation (FIG. 8). Cholesterol was associated mainly with HDL and LDL in apoB-100-only mice and with the Lp(a) fraction in Lp(a)-expressing mice; Lp(a) eluted from a gel filtration column at a position corresponding to a molecular weight greater than LDL in the apoB-100 mice. HDL levels in the Lp(a) mice were similar to those in apoB-100 mice. The triglycerides in Lp(a)-expressing mice were also predominantly in larger-sized particles than in the apoB-100-expressing mice. The increased neutral lipid content in the Lp(a) mice was also apparent from the increased intensity of lipid staining after separation of the plasma lipoproteins by agarose gel electrophoresis (FIG. 8, inset).

FIGS. 8A and 8B. Lipoprotein profiles of mice expressing human apoB-100 or high levels of Lp(a). Pooled plasma from human apoB-100 (n=8) or Lp(a) high-expressing (n=3) mice was size-fractionated by FPLC, and the cholesterol and triglyceride contents of the fractions were determined. Agarose gels of apo(a), apoB-100, and Lp(a) mice showed the different lipid content as detected with Fat Red 7B.

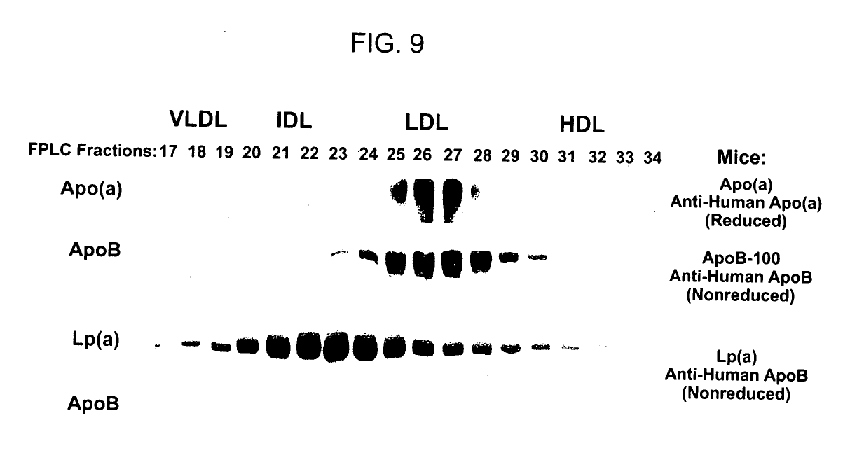

To confirm that the larger lipoproteins in the plasma of Lp(a)-expressing mice were Lp(a), FPLC fractions were separated by SDS-PAGE and examined by immunoblot analysis. As detected by anti-human apo(a), most of the apo(a) in apo(a)-only mice was located in LDL-sized particles (FIG. 9, upper panel). Since these mice lack human apoB-100, apo(a) associates with mouse apoB-100 to form a noncovalent complex to be retained in the LDL fraction. As expected, human apoB-100 in apoB-100 mice was also largely distributed in LDL fractions (FIG. 9, middle panel). Lp(a) in plasma of mice expressing both apo(a) and human apoB-100 was shifted to larger lipoproteins than LDL as shown in the immunoblot with human anti-apoB (FIG. 9, bottom panel). This shift is consistent with the shift observed in the cholesterol and triglyceride distribution profiles from the FPLC columns. In addition, the low amount of free human apoB-100 that was not in the Lp(a) particle was shifted in the same manner, suggesting that a small portion of the apoB-100 associates noncovalently with apo(a) (FIG. 9, bottom panel).

FIG. 9. Distribution of apo(a) and human apoB-100 plasma of transgenic mice expressing either or both proteins. Plasma from mice expressing apo(a), human apoB-100, or Lp(a) was size-fractionated by FPLC, and the fractions were separated by SDS-PAGE, transferred to nitrocellulose, and subjected to immunoblot analysis. FPLC fractions of mouse plasma from the apo(a)-expressing mice were examined under reducing conditions with anti-human apo(a) (upper panel). Fractions of transgenic mouse plasma from mice expressing human apoB-100 (middle panel) or human Lp(a) (lower panel), respectively, were examined under nonreducing conditions with anti-human apoB-100. Formation of a covalent disulfide bond in Lp(a)-expressing mice is indicated by the upper band containing human apoB-100 bound to apo(a). Lp(a) is found in particles larger than apo(a) in apo(a) mice and larger than human apoB-100 in apoB-100 mice.

Oxidized Phospholipid Content of ApoB-100-Containing Lipoproteins

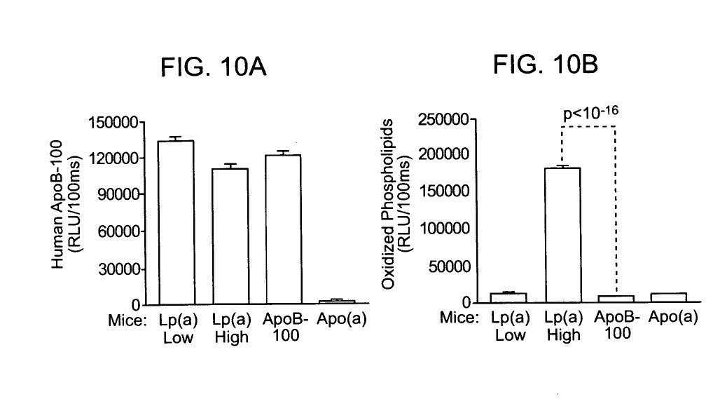

To determine if the lipoproteins from our transgenic mice contained oxidized phospholipids recognized by monoclonal antibody EO6 (24), we examined plasma of mice expressing either human apo(a), human apoB-100, or Lp(a) with a double-antibody sandwich assay. In addition to the Lp(a) high-expressing mice, we also examined plasma of mice expressing low levels of Lp(a) (36±12 mg/dl or 98±32 nmol/l). First human apoB-100-containing lipoproteins were captured from the mouse plasma using MB47, which binds human apoB-100 but not murine apoB-100. The amount of human apoB in mice expressing high levels of Lp(a) was similar to that of mice expressing low levels of Lp(a) or apoB-100 alone (FIG. 10A). As expected, no human apoB-100 was detected in plasma from mice expressing apo(a) alone. The captured human apoB-containing lipoproteins were then assessed for their content of oxidized phospholipid with EO6. A high level of EO6 binding to the apoB-100-containing lipoproteins was noted in mice with high Lp(a) expression, but not in mice expressing low levels of Lp(a) or human apoB-100 alone (FIG. 10B).

FIGS. 10A and 10B. Oxidized phospholipids on captured human apoB-100-containing lipoproteins of transgenic mouse plasma. Human apoB-100-containing lipoproteins were captured from plasma of mice expressing low (n=4) or high (n=5) levels of Lp(a), human apoB-100 (n=9), or human apo(a) (n=8) and examined with chemiluminescence immunoassay for antibody recognition of human apoB-100 (A) and oxidized phospholipids with antibody EO6 (B).

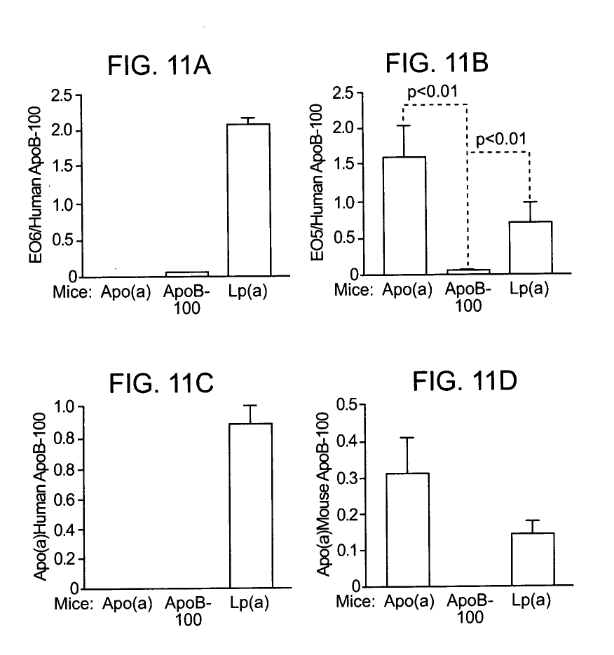

Next, murine apoB-100-containing plasma lipoproteins were captured using monoclonal antibody LF3 and tested for EO6 epitopes. The results are compared with those obtained with human apoB-100 captured with MB47 (FIG. 11A-D). Oxidized phospholipids were detected on the murine apoB containing lipoproteins (FIG. 11B) from mice that expressed apo(a) alone, suggesting that covalent association of apo(a) and apoB is not required for the accumulation or generation of oxidized phospholipids in Lp(a). There was significant association of apo(a) with the captured murine apoB-100 in the apo(a) mice (FIG. 11D). In fact, human apoB-100 was also identified on the murine apoB-100 along with apo(a), demonstrating that intact Lp(a) associated noncovalently with the murine apoB-100. In the Lp(a) mice, there was a relatively lower level of EO6 epitopes on the murine apoB-containing lipoproteins (FIG. 11B), probably because most of the apo(a) recombined with human apoB-100 (FIG. 11A) and was therefore not available for noncovalent association with murine apoB-100. No oxidized phospholipid was found in murine apoB-containing lipoprotein from mice expressing human apoB-100 alone (FIG. 11B). These findings show that oxidized phospholipids detected with EO6 are present almost exclusively on apoB-100 particles to which apo(a) is covalently or even noncovalently bound. However, the oxidized phospholipid content was significantly higher on Lp(a) particles than on human LDL alone (FIG. 11A).

FIG. 11A-D. Oxidized phospholipids and apo(a) in lipoproteins that contains human or mouse apoB-100. The oxidized phospholipid (A) and apo(a) (C) levels of captured human apoB-100-containing lipoproteins are compared with oxidized phospholipid (B) and apo(a) (D) levels of captured mouse apoB-100-containing lipoproteins from plasma of mice expressing human apo(a) (n=8), human apoB-100 (n=10), or high levels of Lp(a) (n=5).

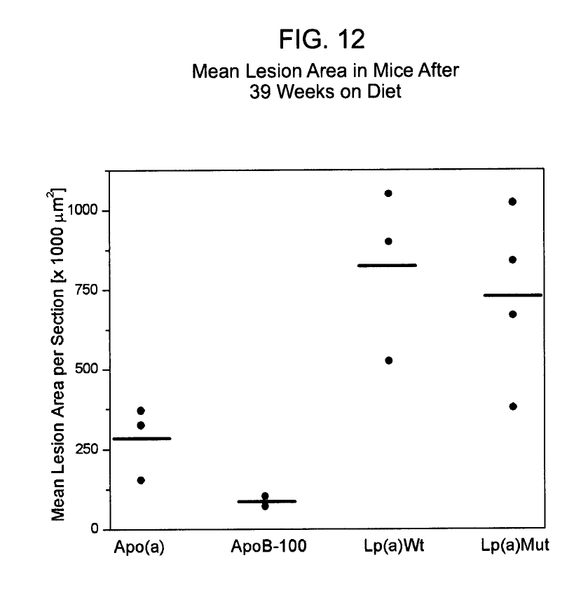

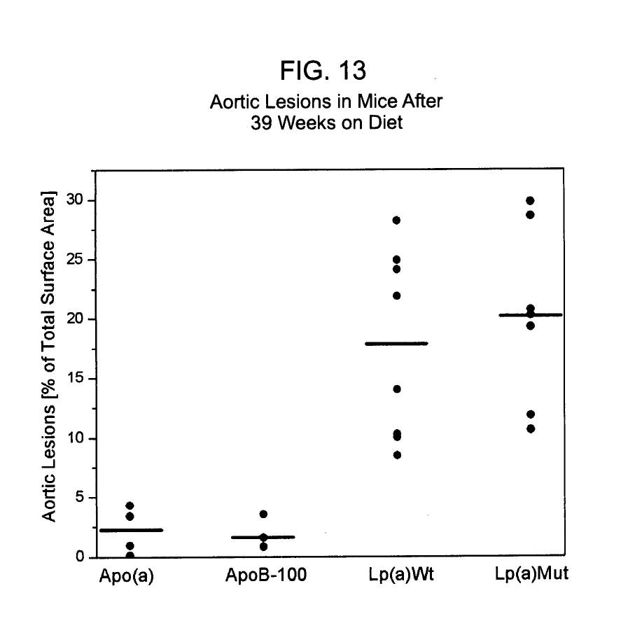

The mean lesion area in the aorta and the aortic lesion (as a percent of total surface area) was determined, in apo(a) single transgenic mice, apoB-100 transgenic mice and Lp(a) mice after 39 weeks on a high fat/high cholesterol diet. The results are shown in FIG. 12 and FIG. 13. FIG. 12 depicts the mean lesion area in aorta of mice after 39 weeks on a high fat, high cholesterol diet. FIG. 13 depicts aortic lesions in mice after 39 weeks on a high fat, high cholesterol diet. The results show that the mean lesion area and the aortic lesions as a percent of total surface area of the aorta was much higher in Lp(a) mice, compared to apo(a) single transgenic mice, and compared to apoB-100 single transgenic mice. These results demonstrate that Lp(a) double transgenic mice display atherosclerotic lesions, and are therefore non-human animal models of atherosclerosis.

### REFERENCES

1. McLean, J. W., Tomlinson, J. E., Kuang, W.-J., Eaton, D. L., Chen, E. Y., Fless, G. M., Scanu, A. M., and Lawn, R. M. (1987) Nature 330, 132-137
2. Bostom, A. G., Cupples, L. A., Jenner, J. L., Ordovas, J. M., Seman, L. J., Wilson, P. W. F., Schaefer, E. J., and Castelli, W. P. (1996) J. Am. Med. Assoc. 276, 544-548
3. Assmann, G., Schulte, H., and von Eckardstein, A. (1996) Am. J. Cardiol. 77, 1179-1184
4. Lawn, R. M., Wade, D. P., Hammer, R. E., Chiesa, G., Verstuyft, J. G., and Rubin, E. M. (1992) Nature 360, 670-672
5. Sanan, D. A., Newland, D. L., Tao, R., Marcovina, S., Wang, J., Mooser, V., Hammer, R. E., and Hobbs, H. H. (1998) Proc. Natl. Acad. Sci. U.S.A. 95, 4544-4549
6. Sun, H., Unoki, H., Wang, X., Liang, J., Ichikawa, T., Arai, Y., Shiomi, M., Marcovina, S. M., Watanabe, T., and Fan, J. (2002) J. Biol. Chem. 277, 47486-47492
7. Fan, J., Shimoyamada, H., Sun, H., Marcovina, S., Honda, K., and Watanabe, T. (2001) Arterioscler. Thromb. Vasc. Biol. 21, 88-94
8. Boonmark, N. W., Lou, X. J., Yang, Z. J., Schwartz, K., Zhang, J.-L., Rubin, E. M., and Lawn, R. M. (1997) J. Clin. Invest. 100, 558-564
9. Fan, J., Wang, J., Bensadoun, A., Lauer, S. J., Dang, Q., Mahley, R. W., and Taylor, J. M. (1994) Proc. Natl. Acad. Sci. U.S.A. 91, 8724-8728
10. Simonet, W. S., Bucay, N., Lauer, S. J., and Taylor, J. M. (1993) J. Biol. Chem. 268, 8221-8229
11. Borén, J., Lee, I., Zhu, W., Arnold, K., Taylor, S., and Innerarity, T. L. (1998) J. Clin. Invest. 101, 1084-1093
12. Sk\dot{a}lén, K., Gustafsson, M., Rydberg, E. K., Hultén, L. M., Wiklund, O., Innerarity, T. L., and Borén, J. (2002) Nature 417, 750-754
13. Marcovina, S. M., Albers, J. J., Gabel, B., Koschinsky, M. L., and Gaur, V. P. (1995) Clin. Chem. 41, 246-255
14. McCormick, S. P. A., Linton, M. F., Hobbs, H. H., Taylor, S., Curtiss, L. K., and Young, S. G. (1994) J. Biol. Chem. 269, 24284-24289
15. Nakajima, K., Hinman, J., Pfaffinger, D., Edelstein, C., and Scanu, A. M. (2001) Arterioscler. Thromb. Vasc. Biol. 21, 1238-1243
16. Shih, D. M., Xia, Y.-R., Wang, X.-P., Miller, E., Castellani, L. W., Subbanagounder, G., Cheroutre, H., Faull, K. F., Berliner, J. A., Witztum, J. L., and Lusis, A. J. (2000) J. Biol. Chem. 275, 17527-17535
17. Svenungsson, E., Jensen-Urstad, K., Heimbürger, M., Silveira, A., Hamsten, A., de Faire, U., Witztum, J. L., and Frosteg\dot{a}rd, J. (2001) Circulation 104, 1887-1893
18. Young, S. G., Witztum, J. L., Casal, D. C., Curtiss, L. K., and Bernstein, S. (1986) Arteriosclerosis 6, 178-188
19. Hörkkö, S., Bird, D. A., Miller, E., Itabe, H., Leitinger, N., Subbanagounder, G., Berliner, J. A., Friedman, P., Dennis, E. A., Curtiss, L. K., Palinski, W., and Witztum, J. L. (1999) J. Clin. Invest. 103, 117-128
20. Friedman, P., Hörkkö, S., Steinberg, D., Witztum, J. L., and Dennis, E. A. (2002) J. Biol. Chem. 277, 7010-7020
21. Zlot, C. H., Flynn, L. M., Véniant, M. M., Kim, E., Raabe, M., McCormick, S. P. A., Ambroziak, P., McEvoy, L. M., and Young, S. G. (1999) J. Lipid Res. 40, 76-84
22. Paultre, F., Pearson, T. A., Weil, H. F. C., Tuck, C. H., Myerson, M., Rubin, J., Francis, C. K., Marx, H. F., Philbin, E. F., Reed, R. G., and Berglund, L. (2000) Arterioscler. Thromb. Vasc. Biol. 20, 2619-2624
23. Kronenberg, F., Kronenberg, M. F., Kiechl, S., Trenkwalder, E., Santer, P., Oberhollenzer, F., Egger, G., Utermann, G., and Willeit, J. (1999) Circulation 100, 1154-1160
24. Tsimikas, S., Bergmark, C., Beyer, R. W., Patel, R., Pattison, J., Miller, E., Juliano, J., and Witztum, J. L. (2003) J. Am. Coll. Cardiol. 41, 360-370
25. Utermann, G. (2001) in The Metabolic and Molecular Bases of Inherited Disease (Scriver, C. R., Beaudet, A. L., Sly, W. S., Valle, D., Childs, B., Kinzler, K. W., and Vogelstein, B., eds) 8th Ed., Vol. 2, pp. 2753-2787, McGraw-Hill, New York
26. Cheesman, E. J., Sharp, R. J., Zlot, C. H., Liu, C. Y.-Y., Taylor, S., Marcovina, S. M., Young, S. G., and McCormick, S. P. A. (2000) J. Biol. Chem. 275, 28195-28200
27. Rader, D. J., Mann, W. A., Cain, W., Kraft, H.-G., Usher, D., Zech, L. A., Hoeg, J. M., Davignon, J., Lupien, P., Grossman, M., Wilson, J. M., and Brewer, H. B., Jr. (1995) J. Clin. Invest. 95, 1403-1408
28. Zioncheck, T. F., Powell, L. M., Rice, G. C., Eaton, D. L., and Lawn, R. M. (1991) J. Clin. Invest. 87, 767-771
29. Ragab, M. S., Selvaraj, P., and Sgoutas, D. S. (1996) Atherosclerosis 123, 103-113
30. Glass, C. K., and Witztum, J. L. (2001) Cell 104, 503-516
31. Schwenke, D. C., and Carew, T. E. (1989) Arteriosclerosis 9, 895-907
32. Williams, K. J., and Tabas, I. (1998) Curr. Opin. Lipidol. 9, 471-474
33. Watson, A. D., Leitinger, N., Navab, M., Faull, K. F., Hörkkö, S., Witztum, J. L., Palinski, W., Schwenke, D., Salomon, R. G., Sha, W., Subbanagounder, G., Fogelman, A. M., and Berliner, J. A.,(1997) J. Biol. Chem. 272, 13597-13607
34. Podrez, E. A., Poliakov, E., Shen, Z., Zhang, R., Deng, Y., Sun, M., Finton, P. J., Shan, L., Gugiu, B., Fox, P. L., Hoff, H. F., Salomon, R. G., and Hazen, S. L. (2002) J. Biol. Chem. 277, 38503-38516
35. Yamada, Y., Doi, T., Hamakubo, T., and Kodama, T. (1998) Cell. Mol. Life Sci. 54, 628-640
36. Gong, Q., and Pitas, R. E. (1995) J. Biol. Chem. 270, 21672-21678
37. Dejager, S., Mietus-Snyder, M., and Pitas, R. E. (1993) Arterioscler. Thromb. 13, 371-378
38. Mietus-Snyder, M., Gowri, M. S., and Pitas, R. E. (2000) J. Biol. Chem. 275, 17661-17670
39. Boullier, A., Gillotte, K. L., Hörkkö, S., Green, S. R., Friedman, P., Dennis, E. A., Witztum, J. L., Steinberg, D., and Quehenberger, O. (2000) J. Biol. Chem. 275, 9163-9169
40. Blencowe, C., Hermetter, A., Kostner, G. M., and Deigner, H. P. (1995) J. Biol. Chem. 270, 31151-31157
41. Berliner, J. A., Subbanagounder, G., Leitinger, N., Watson, A. D., and Vora, D. (2001) Trends Cardiovasc. Med. 11, 142-147
42. Marathe, G. K., Prescott, S. M., Zimmerman, G. A., and McIntyre, T. M. (2001) Trends Cardiovasc. Med. 11, 139-142
43. Chang, M.-K., Bergmark, C., Laurila, A., Hörkkö, S., Han, K.-H., Friedman, P., Dennis, E. A., and Witztum, J. L. (1999) Proc. Natl. Acad. Sci. U.S.A. 96, 6353-6358
44. Hobbs, H. H., and White, A. L. (1999) Curr. Opin. Lipidol. 10, 225-236
45. Dangas, G., Mehran, R., Harpel, P. C., Sharma, S. K., Marcovina, S. M., Dube, G., Ambrose, J. A., and Fallon, J. T. (1998) J. Am. Coll. Cardiol. 32, 2035-2042

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A transgenic non-human animal that comprises, integrated into the genome of the animal, a human apolipoprotein(a) (apo(a)) transgene comprising a nucleotide sequence that encodes apo(a), wherein the nucleotide sequence is operably linked to a promoter element.

2. The transgenic animal of claim 1, wherein said transgene comprises, in order from 5′ to 3′, an apolipoprotein-E (apoE) promoter, an apoE intron, the nucleotide sequence encoding apo(a), and a 3′ hepatic control region.

3. A transgenic non-human animal that comprises, integrated into the genome of the animal, an apolipoprotein(a) (apo(a)) transgene comprising a first nucleotide sequence that encodes apo(a), wherein the first nucleotide sequence is operably linked to a first promoter element; and an apolipoprotein B-100 (apoB-100) transgene comprising a second nucleotide sequence that encodes apoB-100, wherein the second nucleotide sequence is operably linked to a second promoter element, wherein the apo(a) transgene and the apoB-100 transgene are expressed and apo(a) and apoB-100 polypeptides are produced in the animal, wherein a substantial proportion of the apo(a) polypeptides produced are covalently linked to an apoB-100 polypeptide to form lipoprotein(a), and wherein the plasma level of Lp(a) is greater than 30 mg/dL.

4. The transgenic animal of claim 3, wherein the animal develops atherosclerosis when fed a low-fat diet.

5. The transgenic animal of claim 3, wherein the animal develops atherosclerosis when fed a high-fat diet.

6. The transgenic animal of claim 3, wherein the first nucleotide sequence is operably linked to a 3′ hepatic control region.

7. The transgenic animal of claim 3, wherein the first promoter element and the second promoter element are liver specific.

8. The transgenic animal of claim 3, wherein the apo(a) transgene comprises, in order from 5′ to 3′, an apolipoprotein-E (apoE) promoter, an apoE intron, the first nucleotide sequence encoding apo(a), and a 3′ hepatic control region.

9. The transgenic animal of claim 3, wherein the plasma Lp(a) level is greater than 50 mg/dL.

10. The transgenic animal of claim 3, wherein the plasma Lp(a) level is greater than 100 mg/dL.

11. The transgenic animal of claim 3, wherein the animal exhibits plasma triglyceride levels in excess of about 200 mg/dL.

12. The transgenic animal of claim 3, wherein the animal exhibits plasma cholesterol levels in excess of about 200 mg/dL.

13. A method of identifying an agent that treats atherosclerosis, the method comprising:
administering a test agent to the transgenic animal of claim 3; and
determining the effect, if any, of the agent on an atherosclerotic phenotype in the animal.

14. The method of claim 13, wherein said determining is by measuring a plasma level of cholesterol in the animal.

15. The method of claim 13, wherein said determining is by measuring a plasma level of lipoprotein(a) in the animal.

16. The method of claim 13, wherein said determining is by measuring a plasma level of apolipoprotein(a) in the animal.

17. The method of claim 13, wherein said determining is by measuring a plasma level of triglycerides in the animal.

18. The method of claim 13, wherein said determining is by histological examination of the size and/or number of atherosclerotic lesions in an artery from the animal.

19. An isolated polynucleotide that comprises an apolipoprotein(a) (apo(a)) transgene, wherein the apo(a) transgene comprises, in order from 5′ to 3′, an apolipoprotein-E (apoE) promoter, an apoE intron, a nucleotide sequence encoding apo(a), and a 3′ hepatic control region.
